(19) United States
(12) Patent Application Publication
(10) Pub. No.: US 2009/0099259 A1
(43) Pub. Date: Apr. 16, 2009

(54) Method for regulating gene expression

(76) Inventors: Zeina Jouni, Evansville, IN (US); Joshua Anthony, Evansville, IN (US); Steven C. Rumsey, Curitiba (BR); Deshanie Rai, Evansville, IN (US); Kumar Sesha Kothapalli, Ithaca, NY (US); James T. Brenna, Ithaca, NY (US)

Correspondence Address: Richard D. Schmidt;Bristol-Myers Squibb Company, 2400 West Lloyd Expressay, Evansville, IN, 47721-0001, US

(21) Appl. No.: 11/712,102

(22) Filed: Feb. 28, 2007

Related U.S. Application Data
(60) Provisional application No. 60/777,724, filed on Feb. 28, 2006.

(51) Int. Cl. A61K 31/20; A61P 3/00; A61P 25/00; A61P 37/00

(52) U.S. Cl. 514/560

(57) ABSTRACT

The present invention is directed to a novel method for modulating the expression of one or more genes in a subject by administering an amount of DHA and ARA to the subject.

This application claims the priority benefit of U.S. Provisional Application 60/777,724 filed Feb. 28, 2006 which is incorporated by reference herein in its entirety.

### BACKGROUND OF THE INVENTION

(1) Field of the Invention

The present invention relates generally to a method for modulating gene expression in subjects.

(2) Description of the Related Art

Every gene contains the information required to make a protein or a non-coding ribonucleic acid (RNA). In order to produce functional RNA and protein molecules in a cell, however, a gene must be expressed. Gene expression occurs in two major stages, transcription and protein synthesis. During transcription, the gene is copied to produce an RNA molecule (a primary transcript) with essentially the same sequence as the gene. Most human genes are divided into exons and introns, and only the exons carry information required for protein synthesis. Most primary transcripts are therefore processed by splicing to remove intron sequences and generate a mature transcript or messenger RNA (mRNA) that only contains exons.

The second stage of gene expression, protein synthesis, is also known as translation. During this stage there is no direct correspondence between the nucleotide sequence in deoxyribonucleic acid (DNA) and RNA and the sequence of amino acids in the protein. In fact, three nucleotides are required to specify one amino acid.

All genes in the human genome are not expressed in the same manner. Some genes are expressed in all cells all of the time. These so-called housekeeping genes are essential for very basic cellular functions. Other genes are expressed in particular cell types or at particular stages of development. For example, the genes that encode muscle proteins such as actin and myosin are expressed only in muscle cells, not in the cells of the brain. Still other genes can be activated or inhibited by signals circulating in the body, such as hormones.

This differential gene expression is achieved by regulating transcription and translation. All genes are surrounded by DNA sequences that control their expression. Proteins called transcription factors bind to these sequences and can switch the genes on or off. Gene expression is therefore controlled by the availability and activity of different transcription factors.

As transcription factors are proteins themselves, they must also be produced by genes, and these genes must be regulated by other transcription factors. In this way, all genes and proteins can be linked into a regulatory hierarchy starting with the transcription factors present in the egg at the beginning of development. A number of human diseases are known to result from the absence or malfunction of transcription factors and the disruption of gene expression thus caused.

If genes are not expressed in the right time, place and amount, disease may occur. Thus, it would be beneficial to provide a composition that can regulate or modulate the expression of certain genes in subjects and thereby prevent the onset of or treat various diseases and disorders.

### SUMMARY OF THE INVENTION

Briefly, the present invention is directed to a novel method for modulating the expression of one or more genes in a subject, wherein the gene is selected from the group consisting of those genes listed in Tables 4-9 herein under the “Gene Symbol” column, the method comprising administering to the subject DHA and ARA, alone or in combination with one another. The subject can be an infant or a child. The subject can be one that is in need of such modulation. In particular situations, ARA and DHA can be administered in a ratio of ARA:DHA of between about 1:10 to about 10:1 by weight.

The present invention is also directed to a novel method for upregulating the expression of one or more genes in a subject, wherein the gene is selected from the group consisting of those genes listed in Tables 4 and 6 herein under the “Gene Symbol” column, the method comprising administering to the subject DHA or ARA, alone or in combination with one another.

The present invention is additionally directed to a novel method for downregulating the expression of one or more genes in a subject, wherein the gene is selected from the group consisting of those genes listed in Tables 5 and 7 under the “Gene Symbol” column, the method comprising administering to the subject DHA or ARA, alone or in combination with one another.

The present invention is also directed to a novel method for upregulating the expression of one or more genes in a subject, wherein the gene is selected from the group consisting of TIMM8A, TIMM23, NF1, SFTPB, ACADSB, SOD, PDE3A, NSMAF, OSBP2, FTH1, SPTLC2, FOXP2, LUM, BRCA1, ADAM17, ADAM33, TOB1, XCL1, XCL2, RNASE2, RNASE3, SULT1C1, HSPCA, CD44, CD24, OSBPL9, FCER1G, FXD3, NRF1, STK3, and KIR2DS1, the method comprising administering to the subject DHA or ARA, alone or in combination with one another.

The invention is further directed, in an embodiment, to a method for modulating the expression of one or more genes in a subject, wherein the gene is selected from the group consisting of TIMM8A, TIMM23, NF1, LUM, BRCA1, ADAM17, TOB1, RNASE2, RNASE3, NRF1, STK3, FZD3, ADAM8, PERP, COL4A6, PLA2G6, MSRA, CTSD, CTSB, LMX1B, BHMT, TNNC1, PDE3A, PPARD, NPY1R, LEP, and any combination thereof.

The present invention is also, in an embodiment, directed to a method for treating or preventing tumors in a subject, the method comprising modulating a gene selected from the group consisting of TOB1, NF1, FZD3, STK3, BRCA1, NRF1, PERP, and COL4A6 in the subject by administering to the subject an effective amount of DHA or ARA, alone or in combination with one another.

The invention is directed to a method for treating or preventing neurodegeneration in a subject, the method comprising modulating a gene selected from the group consisting of PLA2G6, TIMM8A, ADAM17, TIMM23, MSRA, CTSD, CTSB, LMX1B, and BHMT in the subject by administering to the subject an effective amount of DHA or ARA, alone or in combination with one another. The invention is also directed to a method for improving vision in a subject, the method comprising modulating the LUM gene in the subject by administering to the subject an effective amount of DHA or ARA, alone or in combination with one another. The invention is further directed to a method for treating or preventing macular degeneration in a subject, the method comprising modulating the LUM gene in the subject by administering to the subject an effective amount of DHA or ARA, alone or in combination with one another.

In other embodiments, the invention is directed to a method for stimulating an immune response in a subject, the method comprising modulating a gene selected from the group consisting of RNASE2, RNASE3, and ADAM8 in the subject by administering to the subject an effective amount of DHA or ARA, alone or in combination with one another. The invention is directed to a method for improving lung function in a subject, the method comprising modulating the ADAM33 gene in the subject by administering to the subject an effective amount of DHA or ARA, alone or in combination with one another. The invention is also directed to a method for improving cardiac function in a subject, the method comprising modulating a gene selected from the group consisting of TNNC1 and PDE3A in the subject by administering to the subject an effective amount of DHA or ARA, alone or in combination with one another.

Still further, the invention is directed to a method for treating or preventing obesity in a subject, the method comprising modulating a gene selected from the group consisting of PPARD, NPY1R, and LEP in the subject by administering to the subject an effective amount of DHA or ARA, alone or in combination with one another.

Among the several advantages found to be achieved by the present invention, is that it provides a useful method for the modulation of selected genes in a subject. It also provides a method to upregulate or downregulate certain genes by easily administered compounds. It also provides a method for the prevention and/or treatment of various diseases and disorders in infancy, childhood, adolescence or adulthood.

### BRIEF DESCRIPTION OF THE DRAWINGS

For a more complete understanding of the present invention, reference is now made to the following descriptions taken in conjunction with the accompanying drawings.

FIG. 1 illustrates the ingenuity network analysis generated from L3/C comparisons. The network is graphically represented as nodes (genes) and edges (the biological relationship between genes).

### DETAILED DESCRIPTION OF THE PREFERRED EMBODIMENTS

Reference now will be made in detail to the embodiments of the invention, one or more examples of which are set forth below. Each example is provided by way of explanation of the invention, not a limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

Thus, it is intended that the present invention covers such modifications and variations as come within the scope of the appended claims and their equivalents. Other objects, features and aspects of the present invention are disclosed in or are obvious from the following detailed description. It is to be understood by one of ordinary skill in the art that the present discussion is a description of exemplary embodiments only, and is not intended as limiting the broader aspects of the present invention.

The term “modulation”, as used herein, means a positive or negative regulatory effect on the expression of a gene.

As used herein, the term “upregulate” means a positive regulatory effect on the expression of a gene.

The term “downregulate” means a negative regulatory effect on the expression of a gene.

As used herein the term “expression” means the conversion of genetic information encoded in a gene into mRNA, transfer RNA (tRNA) or ribosomal RNA (rRNA) through transcription.

The term “infant” means a postnatal human that is less than about 1 year of age.

The term “child” means a human that is between about 1 year and 12 years of age. In some embodiments, a child is between the ages of about 1 and 6 years. In other embodiments, a child is between the ages of about 7 and 12 years.

The term “subject” means any animal. Exemplary subjects can be domestic animals, farm or zoo animals, wild animals, non-human animals, or humans. Non-humans subjects can include dogs, cats, horses, pigs, cattle, chickens, turkeys, and the like. Human subjects can be infants, children, and/or adults.

The terms “in need of”, when used to describe a subject, mean that the subject belongs to a class of subjects that would benefit from the gene modulation resulting from the administration of ARA and DHA. In some cases, a subject is in need of such modulation due to genetic factors, and in other cases the subject may be in need of such modulation due to nutritional factors, disease, trauma, or physical disorder.

As used herein, the term “infant formula” means a composition that satisfies the nutrient requirements of an infant by being a substitute for human milk. In the United States, the contents of an infant formula are dictated by the federal regulations set forth at 21 C.F.R. Sections 100, 106, and 107. These regulations define macronutrient, vitamin, mineral, and other ingredient levels in an effort to stimulate the nutritional and other properties of human breast milk.

In accordance with the present invention, the inventors have discovered a novel method for modulating the expression of one or more genes in a subject by administering docosahexaenoic acid (DHA) and arachidonic acid (ARA) to the subject. In some embodiments, certain genes are upregulated and in other embodiments certain genes are downregulated via the method of the present invention. In some embodiments, the method comprises administering docosahexaenoic acid (DHA) and arachidonic acid (ARA) to the subject in a ratio of ARA:DHA of between about 1:10 to about 10:1 by weight. In some embodiments, a ratio of about 1:5 to about 5:1 can be used, and in other embodiments a ratio of about 1:2 to about 2:1 can be used.

In fact, the present inventors have shown that the administration of DHA or ARA, alone or in combination with one another, can modulate the expression of genes across diverse biological processes. They have also shown that DHA or ARA, alone or in combination with one another, modulate the expression of genes involved in learning, memory, speech development, lung function, iron storage and transport, oxygenation, immune function, anti-cancer effects, tumor suppression, adiposity, weight gain, obesity, atherosclerosis and many other biological functions and disorders.

DHA and ARA are long chain polyunsaturated fatty acids (LCPUFA) which have previously been shown to contribute to the health and growth of infants. Specifically, DHA and ARA have been shown to support the development and maintenance of the brain, eyes and nerves of infants. Birch, E., et al., A Randomized Controlled Trial of Long-Chain Polyunsaturated Fatty Acid Supplementation of Formula in Term Infants after Weaning at 6 Weeks of Age, Am. J. Clin. Nutr. 75:570-580 (2002). Clandinin, M., et al., Formulas with Docosahexaenoic Acid (DHA) and Arachidonic Acid (ARA) Promote Better Growth and Development Scores in Very-Low-Birth-Weight Infants (VLBW), Pediatr. Res. 51:187 A-188A (2002). DHA and ARA are typically obtained through breast milk in infants that are breast-fed. In infants that are formula-fed, however, DHA and ARA must be supplemented into the diet.

While it is known that DHA and ARA are beneficial to the development of brain, eyes and nerves in infants, DHA and ARA previously have not been shown to have any effect on the modulation of genetic expression in a subject—in particular in an infant. The effects of DHA or ARA, alone or in combination with one another, on the modulation of genetic expression in the present invention were surprising and unexpected.

In the present invention, the subject can be an infant. Furthermore, the subject can be in need of the modulation of the expression of one or more genes. Such modulation could be upregulation or downregulation of one or more genes. The subject can be at risk for developing a disease or disorder related to the increased or reduced expression of a particular gene. The subject can be at risk due to genetic predisposition, lifestyle, diet, or inherited syndromes, diseases, or disorders.

In the present invention, the form of administration of DHA and ARA is not critical, as long as a therapeutically effective amount is administered to the subject. In some embodiments, the DHA and ARA are administered to a subject via tablets, pills, encapsulations, caplets, gelcaps, capsules, oil drops, or sachets. In another embodiment, the DHA and ARA are added to a food or drink product and consumed. The food or drink product may be a children's nutritional product such as a follow-on formula, growing up milk, or a milk powder or the product may be an infant's nutritional product, such as an infant formula.

When the subject is an infant, it is convenient to provide DHA and ARA as supplements into an infant formula which can then be fed to the infant. The DHA and the ARA can be administered to the subject separately or in combination.

In an embodiment, the infant formula for use in the present invention is nutritionally complete and contains suitable types and amounts of lipid, carbohydrate, protein, vitamins and minerals. The amount of lipid or fat typically can vary from about 3 to about 7 g/100 kcal. The amount of protein typically can vary from about 1 to about 5 g/100 kcal. The amount of carbohydrate typically can vary from about 8 to about 12 g/100 kcal. Protein sources can be any used in the art, e.g., nonfat milk, whey protein, casein, soy protein, hydrolyzed protein, amino acids, and the like. Carbohydrate sources can be any used in the art, e.g., lactose, glucose, corn syrup solids, maltodextrins, sucrose, starch, rice syrup solids, and the like. Lipid sources can be any used in the art, e.g., vegetable oils such as palm oil, canola oil, corn oil, soybean oil, palmolein, coconut oil, medium chain triglyceride oil, high oleic sunflower oil, high oleic safflower oil, and the like.

Conveniently, commercially available infant formula can be used. For example, Enfalac, Enfamil®, Enfamil® Premature Formula, Enfamil® with Iron, Lactofree®, Nutramigen®, Pregestimil®, and ProSobee® (available from Mead Johnson & Company, Evansville, Ind., U.S.A.) may be supplemented with suitable levels of DHA or ARA, alone or in combination with one another, and used in practice of the method of the invention. Additionally, Enfamil® LIPIL®, which contains effective levels of DHA and ARA, is commercially available and may be utilized in the present invention.

The method of the invention requires the administration of a DHA or ARA, alone or in combination with one another. In this embodiment, the weight ratio of ARA:DHA is typically from about 1:3 to about 9:1. In one embodiment of the present invention, this ratio is from about 1:2 to about 4:1. In yet another embodiment, the ratio is from about 2:3 to about 2:1. In one particular embodiment the ratio is about 2:1. In another particular embodiment of the invention, the ratio is about 1:1.5. In other embodiments, the ratio is about 1:1.3. In still other embodiments, the ratio is about 1:1.9. In a particular embodiment, the ratio is about 1.5:1. In a further embodiment, the ratio is about 1.47:1.

In certain embodiments of the invention, the level of DHA is between about 0.0% and 1.00% of fatty acids, by weight. Thus, in certain embodiments, ARA alone may treat or reduce obesity.

The level of DHA may be about 0.32% by weight. In some embodiments, the level of DHA may be about 0.33% by weight. In another embodiment, the level of DHA may be about 0.64% by weight. In another embodiment, the level of DHA may be about 0.67% by weight. In yet another embodiment, the level of DHA may be about 0.96% by weight. In a further embodiment, the level of DHA may be about 1.00% by weight.

In embodiments of the invention, the level of ARA is between 0.0% and 0.67% of fatty acids, by weight. Thus, in certain embodiments of the invention, DHA alone can moderate gene expression in a subject. In another embodiment, the level of ARA may be about 0.67% by weight. In another embodiment, the level of ARA may be about 0.5% by weight. In yet another embodiment, the level of DHA may be between about 0.47% and 0.48% by weight.

The amount of DHA in an embodiment of the present invention is typically from about 3 mg per kg of body weight per day to about 150 mg per kg of body weight per day. In one embodiment of the invention, the amount is from about 6 mg per kg of body weight per day to about 100 mg per kg of body weight per day. In another embodiment the amount is from about 15 mg per kg of body weight per day to about 60 mg per kg of body weight per day.

The amount of ARA in an embodiment of the present invention is typically from about 5 mg per kg of body weight per day to about 150 mg per kg of body weight per day. In one embodiment of this invention, the amount varies from about 10 mg per kg of body weight per day to about 120 mg per kg of body weight per day. In another embodiment, the amount varies from about 15 mg per kg of body weight per day to about 90 mg per kg of body weight per day. In yet another embodiment, the amount varies from about 20 mg per kg of body weight per day to about 60 mg per kg of body weight per day.

The amount of DHA in infant formulas for use in the present invention typically varies from about 2 mg/100 kilocalories (kcal) to about 100 mg/100 kcal. In another embodiment, the amount of DHA varies from about 5 mg/100 kcal to about 75 mg/100 kcal. In yet another embodiment, the amount of DHA varies from about 15 mg/100 kcal to about 60 mg/100 kcal.

The amount of ARA in infant formulas for use in the present invention typically varies from about 4 mg/100 kcal to about 100 mg/100 kcal. In another embodiment, the amount of ARA varies from about 10 mg/100 kcal to about 67 mg/100 kcal. In yet another embodiment, the amount of ARA varies from about 20 mg/100 kcal to about 50 mg/100 kcal. In a particular embodiment, the amount of ARA varies from about 25 mg/100 kcal to about 40 mg/100 kcal. In a particular embodiment, the amount of ARA is about 30 mg/100 kcal.

The infant formula supplemented with oils containing DHA or ARA, alone or in combination with one another, for use in the present invention can be made using standard techniques known in the art. For example, an equivalent amount of an oil which is normally present in infant formula, such as high oleic sunflower oil, may be replaced with DHA or ARA.

The source of the ARA and DHA can be any source known in the art such as marine oil, fish oil, single cell oil, egg yolk lipid, brain lipid, and the like. The DHA and ARA can be in natural form, provided that the remainder of the LCPUFA source does not result in any substantial deleterious effect on the infant. Alternatively, the DHA and ARA can be used in refined form. The LCPUFA source may or may not contain eicosapentaenoic acid (EPA). In some embodiments, the LCPUFA used in the invention contains little or no EPA. For example, in certain embodiments, the infant formulas used herein contain less than about 20 mg/100 kcal EPA; in some embodiments less than about 10 mg/100 kcal EPA; in other embodiments less than about 5 mg/100 kcal EPA; and in still other embodiments substantially no EPA.

Sources of DHA and ARA may be single cell oils as taught in U.S. Pat. Nos. 5,374,657, 5,550,156, and 5,397,591, the disclosures of which are incorporated herein by reference in their entirety.

In an embodiment of the present invention, DHA or ARA, alone or in combination with one another, may be supplemented into the diet of an infant from birth until the infant reaches about one year of age. In a particular embodiment, the infant may be a preterm infant. In another embodiment of the invention, DHA or ARA, alone or in combination with one another, may be supplemented into the diet of a subject from birth until the subject reaches about two years of age. In other embodiments, DHA or ARA, alone or in combination with one another, may be supplemented into the diet of a subject for the lifetime of the subject. Thus, in particular embodiments, the subject may be a child, adolescent, or adult.

In an embodiment, the subject of the invention is a child between the ages of one and six years old. In another embodiment the subject of the invention is a child between the ages of seven and twelve years old. In particular embodiments, the administration of DHA to children between the ages of one and twelve years of age is effective in modulating the expression of various genes, such as those listed in Tables 4-9. In other embodiments, the administration of DHA and ARA to children between the ages of one and twelve years of age is effective in modulating the expression of various genes, such as those listed in Tables 4-9.

In certain embodiments of the invention, DHA or ARA, alone or in combination with one another, are effective in modulating the expression of certain genes in an animal subject. The animal subject can be one that is in need of such regulation. The animal subject is typically a mammal, which can be domestic, farm, zoo, sports, or pet animals, such as dogs, horses, cats, cattle, and the like.

The present invention is also directed to the use of DHA or ARA, alone or in combination with one another, for the preparation of a medicament for modulating the expression of one or more genes in a subject, wherein the gene is selected from the group consisting of those genes listed in Tables 4-7 under the “Gene Symbol” column. In this embodiment, the DHA or ARA, alone or in combination with one another, may be used to prepare a medicament for the regulation of gene expression in any human or animal neonate. For example, the medicament could be used to regulate gene expression in domestic, farm, zoo, sports, or pet animals, such as dogs, horses, cats, cattle, and the like. In some embodiments, the animal is in need of the regulation of gene expression.

The following examples describe various embodiments of the present invention. Other embodiments within the scope of the claims herein will be apparent to one skilled in the art from consideration of the specification or practice of the invention as disclosed herein. It is intended that the specification, together with the examples, be considered to be exemplary only, with the scope and spirit of the invention being indicated by the claims which follow the examples. In the examples, all percentages are given on a weight basis (w/w) unless otherwise indicated.

### Example 1

This example describes the results of DHA and ARA supplementation in modulating gene expression.

### Methods

### Animals

All animal work took place at the Southwest Foundation for Biomedical Research (SFBR) located in San Antonio, Tex. Animal protocols were approved by the SFBR and Cornell University Institutional Animal Care and Use Committee (IACUC). Animal characteristics are summarized in Table 1.

TABLE 1
Baboon Neonate Characteristics

| | |
|---|---|
| Number of animals | 14 |
| Gender | 10 female, 4 male |
| Conceptional age at delivery (days) | 181.8 ± 6.2 |
| Birth weight (g) | 860.3 ± 150.8 |
| Weight at 12 weeks (g) | 1519.1 ± 280.7 |
| Weight gain (g) | 658.8 ± 190.4 |

Fourteen pregnant baboons delivered spontaneously around 182 days gestation. Neonates were transferred to the nursery within 24 hours of birth and randomized to one of three diet groups. Animals were housed in enclosed incubators until 2 weeks of age and then moved to individual stainless steel cages in a controlled access nursery. Room temperatures were maintained at temperatures between 76° F. to 82° F., with a 12 hour light/dark cycle. They were fed experimental formulas until 12 weeks of life.

### Diets

Animals were assigned to one of the three experimental formulas, with LCPUFA concentrations presented in Table 2.

TABLE 2
Formula LCPUFA composition

| | C | L | L3 |
|---|---|---|---|
| DHA (%, w/w) | 0 | 0.42 ± 0.02 | 1.13 ± 0.04 |
| DHA (mg/100 kcal) | 0 | 21.3 ± 1.0 | 62.8 ± 1.9 |
| ARA (%, w/w) | 0 | 0.77 ± 0.02 | 0.71 ± 0.01 |
| ARA (mg/100 kcal) | 0 | 39.4 ± 0.9 | 39.2 ± 0.7 |

Target concentrations were set as shown in brackets and diets were formulated with excess to account for analytical and manufacturing variability and/or possible losses during storage. Control (C) and L, moderate DHA formula, are the commercially available human infant formulas Enfamil® and Enfamil LIPIL®, respectively. Formula L3 had an equivalent concentration of ARA and was targeted at three-fold the concentration of DHA.

Formulas were provided by Mead Johnson & Company (Evansville, Ind.) in ready-to-feed form. Each diet was sealed in cans assigned two different color-codes to mask investigators. Animals were offered 1 ounce of formula four times daily at 07:00, 10:00, 13:00 and 16:00 with an additional feed during the first 2 nights. On day 3 and beyond, neonates were offered 4 ounces total; when they consumed the entire amount, the amount offered was increased in daily 2 ounce increments. Neonates were hand fed for the first 7-10 days until independent feeding was established.

### Growth

Neonatal growth was assessed using body weight measurements, recorded two or three times weekly. Head circumference and crown-rump length data were obtained weekly for each animal. Organ weights were recorded at necropsy at 12 weeks.

### Sampling & Array Hybridization

Twelve week old baboon neonates were anesthetized and euthanized at 84.4±1.1 days. RNA from the precentral gyrus of the cerebral cortex was placed in RNALater according to vendor instructions and was used for the microarray analysis and validation of microarray results.

Microarray studies utilizing baboon samples with human oligonucleotide arrays have been successfully carried out previously. Cerebral cortex global messenger RNA in the three groups was analyzed using Affymetrix Genechip™ HG-U133 Plus 2.0 arrays. See http://www.affymetrix.com/products/arrays/specific/hgu133plus.affx. The HG-U133 Plus 2.0 has >54,000 probe sets representing 47,000 transcripts and variants, including 38,500 well-characterized human genes. One hybridization was performed for each animal (12 chips total). RNA preparations and array hybridizations were processed at Genome Explorations, Memphis, Tenn. <http://www.genome-explorations.com>. The completed raw data sets were downloaded from the Genome Explorations secure ftp servers.

### Statistics

Data are expressed as mean±SD. Statistical analysis was conducted using analysis of variance (ANOVA) to test the hypothesis of equivalent means for measures taken at 12 weeks, and Tukey's correction was used to control for multiple comparisons. Formula consumption, body weight, head circumference, and crown-rump length changes over time were tested with a random coefficient regression model to compare LCPUFA groups (L, L3) to control (C). Analysis were performed using SAS for Windows 9.1 (SAS Institute, Cary, N.C.) with significance declared at p<0.05.

### Microarray Data Analysis

Raw data (.CEL files) were uploaded into Iobion's Gene Traffic MULTI 3.2 (Iobion Informatics, La Jolla, Calif., USA) and analyzed by using the robust multi-array analysis (RMA) method. In general, RMA performs three operations specific to Affymetrix GeneChip arrays: global background normalization, normalization across all of the selected hybridizations, and log2 transformation of “perfect match” oligonucleotide probe values. Statistical analysis using the significance analysis tool set in Gene Traffic was utilized to perform Multiclass ANOVA on all probe level normalized data. Pairwise comparisons were made between C versus L and C versus L3 and all probe set comparisons reaching P<0.05 were included in the analysis. Gene lists of differentially expressed probe sets were generated from this output for functional analysis.

### Bioinformatics Analysis

Expression data was annotated using NIH DAVID <http://apps1.niaid.nih.gov/david> and NetAffx <http://www.affymetrix.com/analysis/index.affx>. Genes were grouped into functional categories and pathways based on the Gene Ontology Consortium <http://www.geneontology.org>, Kyoto Encyclopedia of Genes and Genomes (KEGG) pathway Database <http://www.genome.ip/kegg/pathway.html> and <BioCarta <http://www.biocarta.com/>.

### RNA Isolation and RT PCR

Real-Time Polymerase Chain Reaction (RT PCR) was conducted on nine genes to confirm the results of the array analysis. Total RNA from 30 mg samples of baboon cerebral cortex brain tissue homogenates was extracted using the RNeasy Mini kit (Qiagen, Valencia, Calif.). Each RNA preparation was treated with DNase I according to the manufacturer's instructions. The yield of total RNA was assessed by 260 nm UV absorption. The quality of RNA was analyzed by 260/280 nm ratios of the samples and by agarose gel electrophoresis to verify RNA integrity.

One microgram total RNA from each group (C, L, L3) was reverse-transcribed into first strand cDNA using the iScript cDNA synthesis kit (Bio-Rad, Hercules, Calif.). The iScript reverse transcriptase was a modified MMLV-derived reverse transcriptase and the iScript reaction mix contains both oligo(dT) and random primers. The generated first strand cDNA was stored at −20° C. until used.

Quantitative real-time PCR using SYBR green and TaqMan assay methods was used to verify the differential expression of selected genes that were upregulated in the L3/C comparison. All the primers were gene-specific and generated from human sequences <www.ensembl.org>. PCR primers were designed with PrimerQuest software (IDT, Coralville, Iowa) and ordered from Integrated DNA Technologies (IDT, Coralville, Iowa). Initially primers were tested by polymerase chain reactions with baboon cerebral cortex brain cDNA as template in a 30 μl reaction volume using Eppendorf gradient thermal cycler (Eppendorf), with 1 μm of each primer, 0.25 mm each of dNTPs, 3 μl of 10×PCR buffer (Perkin-Elmer Life Sciences, Foster City, Calif., USA), 1.5 mM MgCl₂ and 1.5 U Taq polymerase (Ampli Taq II; Perkin-Elmer Life Sciences). Thermal cycling conditions were: initial denaturation at 95° C. for 5 minutes followed by 25-35 cycles of denaturation at 95° C. for 30 seconds, annealing at 60° C. for 1 minute and extension at 72° C. for 1 minute, with a final extension at 72° C. for 2 minutes. PCR products were separated by electrophoresis on 2% agarose gel stained with ethidium bromide and bands of appropriate sizes were obtained. The PCR products of LUM, TIMM8A, UCP2, β-ACTIN, ADAM17 and ATP8B1 were sequenced and deposited with GenBank (Acc Numbers: DQ779570, DQ779571, DQ779572, DQ779573, DQ779574 and DQ779575, respectively).

Initially standardized primers for genes (ATP8B1, ADAM17, NF1, FZD3, ZNF611, UCP2, EGFR and control β-ACTIN) were used for SYBR green real time PCR assay (Power SYBR Green PCR Master Mix, Applied Biosystems, Foster City, Calif.). The baboon LUM, TIMM8A and β-ACTIN sequences were used to design TaqMan Assay (Assay by Design; <www.appliedbiosystems.com>). The selected gene symbols, primer pairs and probe details are depicted in Table 3.

TABLE 3

Real Time PCR Primers and Probes TaqMan Assay and SYBR Green Real
Time PCR Primers

| | | |
|---|---|---|
| Gene | | |
| Symbol | Forward Primer | Reverse Primer |

Real Time PCR Primers and Probes TaqMan Assay

| | | | |
|---|---|---|---|
| | | | TaqMan Probe |
| LUM | TGGGCAATCATCACCAAACTGT | ACATGGCACTTGGGTAGCTTT | CAGGGCAGTTACATTCT |
| TIMM8A | TGCACCAGATGACTGAACTTTGTT | AGCCCGACTGTCCAACTTTG | TCCATGCACTTCTCCC |
| β-ACTIN | CCAGCACGATGAAGATCAAGATC | GCCGCCGATCCACACA | CCTGAGCGCAAGTACT |
| | A | | |

SYBR Green Real Time-PCR Primers

| | | |
|---|---|---|
| ATP8B1 | ACCATTGCCTCTGCTCTTGT | TTCAACCGCTTGCGATGCTT |
| ADAM17 | TGCTACTTGCAAAGGCGTGT | ACCCAACGATGTTGTCTGCT |
| NF1 | TGCTGCAATTGCCTGTGTCA | TCCACAACCTTGCACTGCTT |
| FZD3 | ACAGCAGCTTTGGCAATGGA | AATGTGGCCGAGAGGCAAAT |
| ZNF611 | TTGTCAACAGGGCAAGGCAA | TGGGTGCTTCAAGGCCATTT |
| UCP2 | AGCACCGTCAATGCCTACAA | AGGCAGAAGTGAAGTGGCAA |
| EGFR | TGCCAAGGCACGAGTAACAA | AGGAATTCGCTCCACTGTGT |
| β-ACTIN | ATTGCCGACAGGATGCAGAA | AAGCATTTGCGGTGGACGAT |

Quantitative real time PCR reactions were done with the Applied Biosystems Prism 7300/7500 real time PCR system (Applied Biosystems, Foster City, Calif.). After 2 minutes of UNG activation at 50° C., initial denaturation at 95° C. was carried out for 10 minutes, the cycling conditions of 40 cycles consisted of denaturation at 95° C. for 15 seconds, annealing at 60° C. for 30 seconds, and elongation at 72° C. for 1 minute. For SYBR green method UNG activation step was eliminated. All reactions were done in triplicate and β-ACTIN was used as the reference gene. Relative quantification was performed by using comparative CT method (ABI Relative Quantification Chemistry guide # 4347824).

### Network Analysis

A web-delivered bioinformatics tool set, Ingenuity pathway analysis (IPA 3.0)<http://www.ingenuity.com>, was used to identify functional networks influenced by the dietary treatments. IPA is a knowledge database generated from the peer-reviewed scientific publications that enables discovery, visualization and exploration of functional biological networks in gene expression data and delineates the functions most significant to those networks. The 1108 differentially expressed probe sets identified by microarray data, as discussed below, were used for network analyses. Affymetrix probe set ID's were uploaded into IPA and queried against all other genes stored in the IPA knowledge database to generate a set of networks having up to 35 genes. Each Affymetrix probe set ID was mapped to its corresponding gene identifier in the IPA knowledge database. Probe sets representing genes having direct interactions with genes in the IPA knowledge database are called “focus” genes, which were then used as a starting point for generating functional networks. Each generated network was assigned a score according to the number of differentially regulated focus genes in the dataset. These scores are derived from negative logarithm of the P indicative of the likelihood that focus genes found together in a network due to random chance. Scores of 4 or higher have 99.9% confidence level of significance.

### Results and Discussion

Of the 38,000 well-characterized genes analyzed, significance analysis (P<0.05) identified changes in expression levels of approximately 1108 probe sets (ps) in at least one of the brain, spleen, thymus and liver. This represents 2.05% of the total >54,000 ps on the oligoarray. Most ps showed <2-fold change and some genes were modulated differently in different organs.

For the L/C comparisons, 534 ps were upregulated and 574 ps were downregulated, while for the L3/C comparisons, 666 ps were upregulated and 442 ps were downregulated. This illustrates that more genes were overexpressed in the cerebral cortex in response to increasing formula ARA and DHA.

Of the approximately 1108 genes that were modulated, approximately 700 of them have names and known functions. The remaining genes are known only by their license plate (i.e., some ill-described property).

Table 4 illustrates genes that were shown to be upregulated in the brain by DHA and ARA supplementation that have a known biological function. The first column shows the Affymetrix Probe ID No., a number given to the gene during the study. The second column, entitled “Gene Symbol” describes the commonly recognized name of the genes. The third column shows the expression change of the gene. Positive values indicate an upregulation and negative values indicate a downregulation. The expression change is provided as a “log2 value”, or a log base 2 value. For purposes of discussion herein, some of these values were converted to linear percentages.

The fifth column in Table 4, entitled “Organ”, lists an abbreviation for the organ in which the gene was modulated. The abbreviations are as follows: liver (L), brain (B), and thymus (T). The sixth, seventh, eighth and ninth columns, entitled “biological function”, “molecular function”, “cellular component” and “pathway”, provide any known information about that gene related to those functions.

Tables 5 through 7 contain the same categories as those discussed in Table 4. Table 5 illustrates genes that were shown to be downregulated by DHA and ARA supplementation at either 0.33% DHA or 1.00% DHA that have a known biological function. Table 6 illustrates genes that were shown to be upregulated by DHA and ARA supplementation at either 0.33% DHA or 1.00% DHA that have no known biological function. Table 7 illustrates genes that were shown to be downregulated by DHA and ARA supplementation at either 0.33% DHA or 1.00% DHA that have no known biological function.

Table 8 illustrates spleen genes that were either upregulated or downregulated as a result of 1.00% DHA and 0.67% ARA supplementation. The first column shows the Affymetrix Probe ID No., the second column describes the commonly recognized name of the genes, and the third column shows the expression change of the gene. The fourth, fifth, and sixth columns provide any known information about those genes. Table 9 illustrates spleen genes that were either upregulated or down regulated as a result of 0.33% DHA and 0.67% ARA supplementation. The columns are organized in the same manner as those in Table 8.

Tables 4 through 9 are contained on the submitted compact disc and are hereby incorporated by reference in their entireties. The files on the disc are identified as Greenville-#575980-v1-2_{—}9_{—}07_Non-Provisional_Table_{—}4_(19400_.XLS; Size 35,413 KB; Created Feb. 23, 2007; Greenville-#575986-v1-2_{—}9_{—}07_Non-Provisional_Table_{—}5_(19400_{—}.0.XLS; Size 427 KB; Created Feb. 23, 2007; Greenville-#575992-v1-2_{—}9_{—}07_Non-Provisional_Table_{—}6_(19400_{—}.0.XLS; Size 127 KB; Created Feb. 23, 2007; Greenville-#575993-v1-2_{—}9_{—}07_Non-Provisional_Table_{—}7_(19400_{—}.0.XLS; Size 136 KB; Created Feb. 23, 2007; Greenville-#575995-v1-2_{—}9_{—}07_Non-Provisional_Table_{—}8_(19400_{—}.0.XLS; Size 402 KB; Created Feb. 23, 2007; Greenville-#575996-v1-2_{—}9_{—}07_Non-Provisional_Table_{—}9_(19400_{—}.0.XLS; —Size 402 KB; Created Feb. 23, 2007.

Thus, during the early postnatal weeks, supplementation at levels of 0.33% DHA/0.67% ARA (L) and 1.00% DHA/0.67% ARA (L3) altered gene expression across diverse biological processes when compared to an unsupplemented control group. The expression of 1108 genes was altered as a result of DHA/ARA supplementation in the brain tissue, most genes showing less than two-fold changes. When comparing the L group to the C group, 534 genes were upregulated and 574 genes were downregulated. When comparing the L3 group to the C group, 666 genes were upregulated and 442 genes were down-regulated.

Probe sets with ≧1.4 fold expression change are presented in Table 10. Expression change is shown for the L group (third column) as well as the L3 group (fourth column). The L/C comparison corresponds to inclusion of DHA and ARA at current levels near the worldwide breastmilk mean, while the L3 group corresponds to DHA supplementation which is near the worldwide high.

TABLE 10
Probe sets showing ≧1.4 fold changes in gene expression.

| | | Expression change | Expression change |
|---|---|---|---|
| Affymetrix | Gene | (Fold Changes): | (Fold Changes): |
| Probe ID No. | Symbol | L/C | L3/C |
| 231628_s_at | SERPINB6 | −1.45 | 4.59 |
| 231655_x_at | SERPINB6 | | 1.81 |
| 1552719_at | H63 | | 1.64 |
| 1564654_at | COL4A6 | | 1.61 |
| 208137_x_at | ZNF611 | | 1.58 |
| 210800_at | TIMM8A | | 1.56 |
| 233399_x_at | — | | 1.54 |
| 226134_s_at | MSI2 | 1.54 | |
| 224105_x_at | — | | 1.52 |
| 238895_at | TEBP/// | | 1.52 |
| | PTGES3 | | |
| 1560276_at | LOC283403 | | 1.51 |
| 234788_x_at | FLJ13611 | | 1.51 |
| 241867_at | PARP6 | | 1.50 |
| 230867_at | LOC131873 | | 1.50 |
| 212179_at | C6orf111 | | 1.49 |
| 205745_x_at | ADAM17 | | 1.49 |
| 233808_at | STK3 | | 1.48 |
| 242273_at | — | | 1.48 |
| 216051_x_at | — | | 1.48 |
| 1553844_a_at | C10orf67 | | 1.47 |
| 214768_x_at | — | | 1.47 |
| 215604_x_at | — | | 1.47 |
| 215208_x_at | RPL35A | | 1.46 |
| 242391_at | — | | 1.45 |
| 224143_at | TTTY8 | | 1.45 |
| 239199_at | — | | 1.45 |
| 238269_at | FBXL7 | | 1.44 |
| 231538_at | C11orf1 | | 1.44 |
| 244310_at | — | | 1.44 |
| 208844_at | VDAC3 | | 1.43 |
| 221304_at | UGT1A10 | | 1.42 |
| 235767_x_at | PHAX | | 1.42 |
| 234652_at | — | | 1.41 |
| 1554583_a_at | MGC50559 | | 1.41 |
| 216600_x_at | ALDOB | | 1.41 |
| 235425_at | SGOL2 | | 1.41 |
| 242016_at | LOC284409 | 1.40 | |
| 216202_s_at | SPTLC2 | | 1.40 |
| 234594_at | C14orf85 | | 1.40 |
| 233868_x_at | ADAM33 | | 1.40 |
| 227149_at | TNRC6C | 1.40 | |
| 1553641_a_at | TSGA13 | | −1.40 |
| 218575_at | ANAPC1 | | −1.41 |
| 234006_s_at | RP4-622L5 | | −1.41 |
| 229023_at | MGC5391 | | −1.41 |
| 203023_at | HSPC111 | | −1.41 |
| 216300_x_at | RARA | | −1.41 |
| 221418_s_at | THRAP5 | | −1.41 |
| 244858_at | TGIF | | −1.42 |
| 210764_s_at | CYR61 | | −1.42 |
| 223852_s_at | MGC4796 | | −1.42 |
| 206681_x_at | GP2 | | −1.42 |
| 222349_x_at | RNF126P1 | | −1.43 |
| 230117_at | LOC285878 | −1.44 | |
| 229118_at | PRRG3 | | −1.44 |
| 219088_s_at | ZNF576 | | −1.44 |
| 241399_at | FAM19A2 | | −1.44 |
| 209364_at | BAD | | −1.45 |
| 202862_at | FAH | | −1.47 |
| 244128_x_at | GLIS1 | | −1.48 |
| 205839_s_at | BZRAP1 | | −1.49 |
| 211534_x_at | PTPRN2 | | −1.49 |
| 221256_s_at | HDHD3 | | −1.49 |
| 223018_at | NOB1P | | −1.50 |
| 204647_at | HOMER3 | | −1.50 |
| 224451_x_at | ARHGAP9 | | −1.54 |
| 205440_s_at | NPY1R | −1.56 | |
| 237847_at | — | | −1.56 |
| 238996_x_at | ALDOA | | −1.62 |
| 203395_s_at | HES1 | | −1.63 |
| 220551_at | SLC17A6 | −3.21 | −1.87 |

Nine genes were tested by quantitative real time PCR to confirm the array results, as shown in Table 11. All were qualitatively consistent with the gene array results.

TABLE 11
Comparison of microarray versus QRT-PCR
gene expression values (Fold-changes)

| | | QRT- | | QRT- |
|---|---|---|---|---|
| | Microarray | PCR | Microarray | PCR |
| Gene Name | L/C | L/C | L3/C | L3/C |
| Lumicam (LUM) | 1.03 | 4.18 | 1.30 | 6.04 |
| Translocase of inner | 1.04 | 1.33 | 1.57 | 1.76 |
| mitochondrial membrane 8 | | | | |
| homolog A (TIMM8A) | | | | |
| ATPase, Class I, type 8B, | 1.28 | 1.13 | 1.36 | 1.20 |
| member 1 (ATP8B1) | | | | |
| ADAM metallopeptidase | 1.37 | 1.95 | 1.50 | 2.66 |
| domain 17 (ADAM17) | | | | |
| Neurofibromin 1 (NF1) | 1.03 | 2.07 | 1.20 | 2.16 |
| Frizzled homolog 3 (FZD3) | 1.13 | 1.80 | 1.20 | 2.58 |
| Zinc finger protein 611 | 1.10 | 1.46 | 1.60 | 3.25 |
| (ZNF611) | | | | |
| Uncoupling protein 2 (UCP2) | 1.16 | 2.18 | 1.30 | 3.82 |
| Epidermal growth factor | −1.08 | −1.20 | 1.17 | 1.40 |
| receptor (EGFR) | | | | |

Functional characterization by gene ontology of these differentially regulated genes assigns them to diverse biological processes including lipid and other metabolism, ion channel and transport, development, visual perception, G-protein and signal transduction, regulation of transcription, cell cycle, cell proliferation, and apoptosis. Several categories of gene ontogeny which were influenced by DHA and ARA supplementation are discussed below.

### Lipid (Fatty Acid and Cholesterol) Metabolism

Table 12 presents results from genes related to lipid metabolism that are regulated by dietary LCPUFA.

TABLE 12
Lipid and energy metabolism gene modulation
in expression profiles.

| | Gene | | | |
|---|---|---|---|---|
| Metabolism | Symbol | Unigene ID | L1 | L3 |
| Lipid | ATP8B1 | Hs.569910 | 1.28 | 1.36 |
| | PDE3A | Hs.386791 | 1.08 | 1.30 |
| | ELOVL5 | Hs.520189 | −1.02 | 1.11 |
| | ACSL3 | Hs.471461 | −1.13 | 1.08 |
| | HNF4A | Hs.116462 | 1.06 | −1.16 |
| | CLPS | Hs.1340 | 1.02 | −1.16 |
| | ALDH3B2 | Hs.87539 | 1.05 | −1.16 |
| | PLCE1 | Hs.20022 | −1.10 | −1.19 |
| Fatty acid | ACADSB | Hs.81934 | −1.10 | 1.38 |
| oxidation | ACAD10 | Hs.331141 | −1.08 | 1.10 |
| | GLYAT | Hs.274336 | 1.01 | 1.30 |
| | ADH5 | Hs.78989 | 1.03 | 1.22 |
| | CPT2 | Hs.145384 | 1.10 | −1.22 |
| Energy | LEP | Hs.194236 | −1.01 | 1.17 |
| Ceramide | NSMAF | Hs.372000 | −1.04 | 1.31 |
| | LASS5 | Hs.270525 | 1.06 | 1.11 |
| Glycosphingolipid | SPTLC2 | Hs.435661 | 1.27 | 1.40 |
| Steroid | OSBP2 | Hs.517546 | −1.17 | 1.35 |
| | UGT2B15 | Hs.150207 | 1.04 | 1.21 |
| | SULT2B1 | Hs.369331 | 1.04 | −1.38 |
| Phospholipid | DGKE | Hs.546318 | −1.10 | 1.17 |
| | PLA2G6 | Hs.170479 | −1.09 | −1.20 |
| Prostaglandin | TEBP | Hs.50425 | 1.02 | 1.52 |
| and Leukotriene | ANXA3 | Hs.480042 | 1.26 | −1.04 |
| | LTC4S | Hs.456 | −1.33 | −1.24 |
| Cholesterol | DHCR24 | Hs.498727 | −1.18 | 1.17 |
| | PRKAG2 | Hs.131133 | −1.07 | 1.09 |
| | PRKAA1 | Hs.43322 | 1.09 | −1.02 |
| | SOAT1 | Hs.496383 | −1.09 | −1.12 |
| | FDFT1 | Hs.546253 | 1.01 | −1.13 |

Genes related to phospholipids biosynthesis (PLA2G6 and DGKE) were differentially expressed. PLA2G6 was downregulated in both groups. This gene codes for the Ca-independent cytosolic phospholipase A2 Group VI. Alterations in this gene have very recently been implicated as a common feature of neurodegenerative disorders involving iron accumulation, Morgan, N. V., et al., PLA2G6, Encoding a Phospholipase A2, is Mutated in Neurodegenerative Disorders with High Brian Iron, Nat. Genet. 38(7): 752-54 (2006), as well as the underlying factor in infantile neuroaxonal dystrophy, a neurodegenerative disorder caused by accumulation of iron in the globus pallidus and resulting in death by age 10. Khateeb, S., et al., PLA2G6 Mutation Underlies Infantile Neuroaxonal Dystrophy, Am. J. Hum. Genet. 79(5): 942-48 (2006). PLA2 are a superfamily of enzymes that liberate fatty acids from the sn-2 position of phospholipids; in the globus pallidus DHA and ARA are the most abundant acyl groups at this site. Thus, the present invention has shown to be useful in downregulating PLA2G6, thereby preventing or treating neurodegerative disorders.

Remarkably, among the elongation and desaturation enzymes associated with LCPUFA synthesis, only a single elongation enzyme was differentially expressed. The human ELOVL5 transcript was downregulated slightly in the L/C group and upregulated in the L3/C group. This enzyme, also called HELO1, catalyzes the two carbon elongation of polyunsaturated 18 and 20 carbon fatty acids. Leonard, A. E., et al., Cloning of a Human cDNA Encoding a Novel Enzyme Involved in the Elongation of Long-Chain Polyunsaturated Fatty Acids, Biochem. J. 350 Pt. 3: 765-70 (2000); Leonard, A. E., et al., Identification and Expression of Mammalian Long-Chain PUFA Elongation Enzymes, Lipids 37(8): 733-40 (2002).

The inventors also found that DGKE was upregulated in the L3/C comparison. Genes involved in ceramide metabolism (NSMAF, LASS5), glycosphingolipid metabolism (SPTLC2) and steroid metabolism (OSBP2, UGT2B15) showed increased expression in L3/C group, whereas NSMAF and OSBP2 were downregulated in L/C group.

A further gene modulated by DHA and ARA supplementation was serine palmitoyltransferase, long-chain base subunit 2 (SPTLC2). Serine palmitoyl-CoA transferase (SPT) is the key rate-limiting enzyme in the biosynthesis of sphingolipids. Sphingolipids play a very important role in cell membrane formation, signal transduction, and plasma lipoprotein metabolism. SPT is considered to be a heterodimer of two subunits of Sptlc1 and Sptlc2. A SPTLC2 deficiency causes a significant decrease in plasma Ceramide levels. Ceramide is a well known second messenger and plays an important role in apoptosis. Strategies elevating cellular Ceramide are employed for therapies aimed at arresting growth or promoting apoptosis. M. R. Hojjati, et al., Serine Palmitoyl-CoA Transferase (SPT) Deficiency and Sphingolipid Levels in Mice, Biochim Biophys Acta. 1737(1):44-51 (2005); Y. A. Hannun, et al., Enzymes of Sphingolipid Metabolism: From Modular to Integrative Signaling, Biochemistry 40(16):4893-903 (2001).

A SPTLC2 deficiency causes a significant decrease of plasma S1P (sphingosine-1-phosphate) levels. In human plasma, 65% of S1P is associated with lipoproteins, where HDL is the major carrier. The S1P in HDL has been shown to bind to S1P/Edg receptors on human endothelial cells, and for this reason is believed to mediate many of the anti-inflammatory actions of HDL on endothelial cells. F. Okajima, Plasma Lipoproteins Behave as Carriers of Extracellular Sphingosine 1-Phosphate: Is this an Atherogenic Mediator or an Anti-Atherogenic Mediator? Biochim Biophy. Acta. 1582:132-137 (2002); T. Kimura, et al., High-Density Lipoprotein Stimulates Endothelial Cell Migration and Survival Through Sphingosine 1-Phosphate and its Receptors. Arterioscler Thromb Vasc Biol. 23:1283-1288 (2003).

A SPTLC2 deficiency also causes dramatically decreased plasma LysoSM (lysosphingomyelin) levels. LysoSM is a putative second messenger important in several intracellular and intercellular events, and has been implicated in regulation of cell growth, differentiation, and apoptosis. It increases intracellular calcium concentration and nitric oxide production in endothelial cells, causing endothelium-dependent vasorelaxation of bovine coronary arteries. Y. Xu. Sphingosylphosphorylcholine and Lysophosphatidylcholine: G Protein-Coupled Receptors and Receptor-Mediated Signal Transduction. Biochim Biophys Acta. 1582:81-88 (2002); K. Mogami, et al., Sphingosylphosphorylcholine Induces Cytosolic Ca(2+) Elevation in Endothelial Cells in Situ and Causes Endothelium-Dependent Relaxation through Nitric Oxide Production in Bovine Coronary Artery. FEBS Lett. 457:375-380 (1999).

As shown in Table 9, SPTLC2 was upregulated in both the L group and the L3 group in the present study. It is believed that supplementation with DHA and ARA can increase plasma LysoSM levels and plasma S1P levels.

The best studied role of ARA is as a precursor for eicosanoids including prostaglandins, leukotrienes, and thromboxanes. One of the genes derived from membrane-bound ARA, which catalyze the first step in the biosynthesis of cysteinyl leukotrienes, Leukotriene C4 synthase (LTC4S), was downregulated in both DHA/ARA groups. LTC4S is a potent proinflammatory and anaphylactic mediator. Welsch, D. J., et al, Molecular Cloning and Expression of Human Leukotriene-C4 Synthase, Proc. Natl. Acad. Sci. 91(21): 9745-49 (1994). Thus, it is believed that DHA and ARA supplementation may have anti-inflammatory effects due to its downregulation of LTC4S.

An elevated level of mRNA for PGES3 (prostaglandin E synthase 3) was observed in both of the feeding groups. PGES3 is also known as TEBP (telomerase-binding protein p23) or inactive progesterone receptor, 23-KD (p23). A ubiquitous highly conserved protein which functions as a co-chaperone for the heat shock protein, HSP90, p23 participates in the folding of a number of cell regulatory proteins. Buchner, J., Hsp90 & Co.—A Holding for Folding, Trends Biochem. Sci. 24(4): 136-41 (1999); Weaver, A. J., et al., Crystal Structure and Activity of Human p23, a Heat Shock Protein 90 Co-Chaperone, J. Bio. Chem. 275(30): 23045-52 (2000). It has been demonstrated to bind to human telomerase reverse transcriptase (hTERT) and contribute to telomerase activity. Holt, S. E., et al., Functional Requirement of p23 and Hsp90 in Telomerase Complexes, Genes Dev. 13(7): 817-26 (1999). Decreased levels of Annexin A3 (ANXA3) also known as Lipocortin III was observed with increasing DHA.

Genes involved in fatty acid oxidation (ACADSB, ACAD10 and GLYAT) were overexpressed and carnitine palmitoyltransferase II (CPT2) downregulated in the L3/C group. The upregulation of both the ACADs family members ACADSB and ACAD10 in the L3/C group was consistent with greater energy production in the high DHA group. ACADs (acyl-CoA dehydrogenases) are a family of mitochondrial matrix flavoproteins that catalyze the dehydrogenation of acyl-CoA derivatives and are involved in the β-oxidation and branched chain amino-acid metabolism. Rozen, R. et al., Isolation and Expression of a cDNA Encoding the Precursor for a Novel Member (ACADSB) of the acyl-CoA Dehydrogenase Gene Family, Genomics 24(2):280-87 (1994); Ye, X., et al., Cloning and Characterization of a Human cDNA ACAD10 Mapped to Chromosome 12q24.1, Mol. Bio. Rep. 31(3): 191-95 (2004). ACADSB deficiency causes isolated 2-methylbutyrylglycinuria, a defect in isoleucine catabolism. Isolated excretion of 2-methylbutyrylglycine (2-MBG), a recently identified defect in the proximal pathway of L-isoleucine oxidation, is caused by ACADSB deficiency.

Mitochondrial-specific GLYAT (glycine-N-acyltransferase) also known as acyl CoA:glycine N-acyl transferase (ACGNAT), conjugates glycine with acyl-CoA and participates in detoxification of various drugs and xenobiotics. Mawal, Y. R. & Qureshi, I. A., Purification to Homogeneity of Mitochondrial Acyl coa:glycine n-acyltransferase from Human Liver, Biochem. Biophys. Res. Commun. 205(2): 1373-79 (1994); Mawal, Y. R., et al., Developmental Profile of Mictochondrial Glycine N-Acyltransferase in Human Liver, J. Pediatr. 130(6): 1003-7 (1997). Mawal, et al. also suggested that delayed development of GLYAT might impair detoxification process in children.

Genes involved in cholesterol biosynthesis, DHCR24, PRKAG2, PRKAA1, SOAT1, and FDFT1 showed significant associations with LCPUFA levels. Increasing DHA upregulated DHCR24 and PRKAG2 and downregulated PRKAA1, SOAT1 and FDFT1. DHCR24 (24-dehydrocholesterol reductase), also known as selective AD indicator 1 (SELADIN1), catalyzes the reduction of the A-24 double bond of sterol intermediates during cholesterol biosynthesis. Waterham, H. R., et al., Mutations in the 3beta-Hydroxysterol Delta-Reductase Gene Cause Desmosterolosis, An Autosomal recessive Disorder of Cholesterol Biosynthesis, Am. J. Hum. Genet. 69(4): 985-94 (2001). SELADIN1 may activate estrogen receptors in the brain and protect from beta-amyloid-mediated toxicity. Peri, A. G., et al., Seladin-1 as a Target of Estrogen Receptor Activation in the Brain: A New Gene for a Rather Old Story? J. Endocrin. Invest. 28(3): 285-93 (2005). Decreased expression of SELADIN1 was observed in brain regions of patients with Alzheimer's disease. Benvenuti, S., et al., Estrogen and Selective Estrogen Receptor Modulators Exert Neuroprotective Effects and Stimulate the Expression of Selective Alzheimer's Disease Indicator-1, A Recently Discovered AntiApoptotic Gene, in Human Neuroblast Long-Term Cell Cultures, J. Clin. Endocrin. Metab. 90(3): 1775-82 (2005). PRKAG2 (protein kinase, AMP-activated, gamma 2) is a member of AMP-activated protein kinase (AMPK) family. AMPKs perform multifunctional roles in calcium signaling, weight loss, regulation of energy metabolism in heart. Evans, A. M., AMP-Activated Protein Kinase and the Regulation of Ca2+ Signalling in O2-Sensing Cells, J. Physiol. (2006); Watt, M. J., et al., CNTF Reverses Obesity-Induced Insulin Resistance by Activating Skeletal Muscle AMPK, Nat. Med. 12(5): 541-48 (2006); Dyck, J. R., et al., AMPK Alterations in Cardiac Physiology and Pathology: Enemy or Ally? J. Physiol. (2006).

SOAT1 (sterol O-acyl transferase) or Acyl-coenzyme A:cholesterol acyl transferase (ACAT) is an intracellular protein which catalyzes the formation of cholesterol esters in endoplasmic reticulum and is involved in lipid droplets that are characteristic of foam cells of atherosclerotic plaques. Miyazaki, A., et al., Inhibitors of Acyl-CoEnzyme A:Cholesterol Acyltransferase, Curr. Drug Targets Cardio. Haematol. Disorder, 5(6): 463-69 (2005); Stein, O. & Stein, Y., Lipid Transfer Protein (LTP) and Atherosclerosis, Pharm. Res. 22(10) 1578-88 (2005); Leon, C., et al., Potential Role of Acyl-Coenzyme A:Cholesterol Transferase (ACAT) Inhibitors as Hypolipidemic and Antiatherosclerosis Drugs, Pharm. Res. 22(10) 1578-88 (2005).

Increased expression was detected for ATP8B1 and PDE3A in both groups, comparatively more in L3/C, while transcripts involving HNF4A (Hepatic nuclear factor-4-α), CLPS, and ALDH3B2 showed decreased expression with increasing DHA. ATP8B1 expression was confirmed by real time PCR.

Intrahepatic cholestasis, or impairment of bile flow, is an important manifestation of inherited and acquired liver disease resulting in hepatic accumulation of the toxic bile acids and progressive liver damage. Bile acids enhance efficient digestion and absorption of dietary fats and fat-soluble vitamins, and are the main route for excretion of sterols. Expression of ATP8B1 is high in the small intestine, and mutations in the ATP8B1 gene have been linked to intrahepatic cholestasis. Bull, L. N., et al., A Gene Encoding a P-Type ATPase Mutated in Two Forms of Hereditary Cholestasis, Nat. Genet. 18(3): 219-24 (1998); Mullenbach, R., et al., ATP8B1 Mutations in British Cases with Intrahepatic Cholestasis of Pregnancy, Gut. 54(6): 829-34 (2005). ATP8B1 may function as a bile salt transporter. The knockout mouse phenotype of ATP8B1 revealed a disruption in bile salt homeostasis without impairment of bile secretion. Calcium malabsorption, magnesium deficiency and vitamin D deficiency are often associated with osteoporosis and hypocalcemia in cholestatic liver diseases. It has been suggested that the ATP8B1 gene is involved in gene calcium regulation via the parathyroid hormone.

PDE3A (phosphodiesterase 3A, cGMP-inhibited) is a 120 kDa protein found in myocardium and platelets. Liu, H., Expression of Cyclic GMP-Inhibited Phosphodiesterases 3A and 3B (PDE3A and PDE3B) in Rat Tissues Differential Subcellular Localization and Regulated Expression by Cyclic AMP, Br. J. Pharm. 125(7): 1501-10 (1998). Ding, et al. showed significantly decreased expression of PDE3A in the left ventricles of failing human hearts. Ding, B., et al., Functional Role of Phosphodiesterase 3 in Cardiomyocyte Apoptosis: Implication in Heart Failure, Circulation 111(19): 108-14 (2000). Genetic evidence indicates that resumption of meiosis in vivo and in vitro requires PDE3A activity. Complete sterility was noted in female PDE3A−/− mice. PDE3A expression also is required for the regulation of penile erection in humans. Kuthe, A., et al., Gene Expression of the Phosphodiesterase 3A and 5A in Human Corpus Cavernosum Penis, Eur. Urol. 38(1): 108-14 (2000).

Leptin (LEP), which has a role in energy metabolism, was overexpressed in the brain tissue of the L3/C group. Leptin is a secreted adipocyte hormone that plays a pivotal role in the regulation of food intake and energy homeostasis. Zhang, Y., et al., Positional Cloning of the Mouse Obese Gene and Its Human Homologue, Nature 372(6549):543-46 (1995); Halaas, J. L., et al., Weight-Reducing Effects of the Plasma Protein Encoded by the Obese Gene, Science 269(5223): 543-46 (1995). Leptin suppresses feeding and decreases adiposity in part by inhibiting hypothalamic Neuropeptide Y synthesis and secretion. Stephens, T. W., et al., The Role of Neuropeptide Y in the Antiobesity Action of the Obese Gene Product, Nature 377(6549) 530-32 (1995); Schwartz, M. W., et al., Identification of Targets of Leptin Action in Rat Hypothalamus, J. Clin. Invest. 98(5): 1101-06 (1996). In diabetic mice, administration of LEP reduced hyperphagia, hyperglycemia, and Ghrelin mRNA levels. Decreased mRNA levels of LEP were detected in obese mice.

Based on the modulation of the above-noted genes, the inventors have shown that DHA and ARA are useful in altering lipid metabolism. More specifically, DHA and ARA supplementation may provide greater energy production, regulation of energy metabolism, suppression of appetite, and weight loss. Accordingly, in an embodiment, the present invention is directed to a method for improving body composition in a subject by administering a therapeutically effective amount of DHA and ARA to that subject.

### Ion Channel and Transport

Expression levels of transcripts involved in ion channel and transporter activity were altered by dietary LCPUFA. Uncoupling protein 2 LOC131873 (hypothetical protein) and ATP11C, which have ion channel activity, are upregulated in both the groups but more so in L3/C. Other transcripts with ion channel activity, including VDAC3, FTH1, KCNK3, KCNH7, and TRPM1 were overexpressed in L3/C group and underexpressed in L/C. GLRA2, TRPV2 and HFE are overexpressed in L/C and repressed in L3/C. P2RX2, GRIA1 and CACNA1S are repressed in both the groups.

One of the significant observations in the present invention is the overexpression of uncoupling protein 2 (UCP2), a mitochondrial proton carrier. The data shows an increased expression of UCP2 in neonatal cerebral cortex associated with dietary LCPUFA; increased expression was observed in both the groups but more so in L3/C. QRT-PCR confirmed the array results. Nutritional regulation and induction of mitochondrial uncoupling proteins resulting from dietary n3-PUFA in skeletal muscle and white adipose tissue have been observed. Baillie, R. A., et al., Coordinate Induction of Peroxisomal Acyl-CoA Oxidase and UCP-3 by Dietary Fish Oil: A Mechanism for Decreased Body Fat Deposition, Prostaglandins Leukot. Essent. Fatty Acids, 60(5-6): 351-56 (1999); Hun, C. S., et al., Increased Uncoupling Protein2 mRNA in White Adipose Tissue, and Decrease in Leptin, Visceral Fat, Blood Glucose, and Cholesterol in KK-Ay Mice Fed with Eicosapentaenoic and Docosahexaenoic Acids in Addition to Linolenic Acid, Biochem. Biophys. Res. Commun. 259(1): 85-90 (1999). Increased UCP2 expression is beneficial in diseases associated with neurodegeneration, cardiovascular and type-2 diabetes. Mattiasson, G. & Sullivan, P. G., The Emerging Functions of UCP2 in Health, Disease, and Therapeutics, Antixoid. Redox Signal, 8(1-2) 1-38 (2006). Dietary fats in milk increased the expression and function of UCP2 in neonatal brain and protected neurons from excitotoxicity. Sullivan, P. G., et al., Mitochondrial Uncoupling Protein-2 Protects the Immature Brain from Excitotoxic Nueronal Death, Ann. Neurol. 53(6): 711-717 (2003).

VDAC3 (voltage-dependent anion channel 3) belongs to a group of pore forming proteins found in the outer mitochondrial membrane and in brain synaptic membranes. Blachly-Dyson, E., et al., Human Genes Encoding the Voltage-Dependent Anion Channel (VDAC) of the Outer MitoChondrial Membrane: Mapping and Identification of Two New Isoforms, Geomics 20(1): 62-67 (1994); Shafir, I., et al., Voltage-Dependent Anion Channel Proteins in Synaptosomes of the Torpedo Electric Organ Immunolocalization, Purification, and Characterization, J. Bioenerg. Biomembr. 30(5): 499-510 (1998). Massa, et al., observed a significant reduction of VDAC3 mRNA levels in the skeletal muscle and brains of dystrophin-deficient mdx mice during postnatal development. Massa, R., et al., Intracellular Localization and Isoform Expression of the Voltage-Dependent Anion Channel (VDAC) in Normal and Dystrophic Skeletal Muscle, J. Muscle Res. Cell. Motil. 21(5): 433-42 (2000). Mice lacking VDAC3 exhibit infertility. Sampson, M. J., et al., Immotile Sperm and Infertility in Mice Lacking Mitochondrial Voltage-Dependent Anion Channel Type 3, J. Biol. Chem. 276(42): 39206-12 (2001). All the transcripts (VDAC3, KCNK3 and KCNH7) having voltage-gated anion channel porin activity were overexpressed with increasing DHA.

The present invention has shown that FTH1 (ferritin heavy chain 1) is upregulated by DHA and ARA supplementation in infancy. FTH1 is the primary iron storage factor and is required for iron homeostasis. It has been previously shown to be expressed in the human brain. Percy, M. E., et al., Iron Metabolism and Human Ferritin Heavy Chain cDNA from Adult Brain with an Elongated Untranslated Region: New Findings and Insights, Analyst 123(1): 41-50 (1998). It has been identified as an essential mediator of the antioxidant and protective activities of NF-κB. A reduced expression of FTH1 may be responsible for abnormal accumulation of ferritin and may be responsible for human cases of hyperferritenemia. Abnormal accumulation of ferritin was found to be associated with an autosomal dominant slowly progressing neurodegenerative disease clinically characterized by tremor, cerebellar ataxia, Parkinsonism, pyramidal signs, behavioral disturbances, and cognitive decline. FTH1 was downregulated in the L group by 8%, but was upregulated in the L3 group by 37%, as compared to the control group. Thus, it is believed that the upregulation of FTH1 by DHA and ARA supplementation in infancy can improve iron absorption and/or can prevent the onset of various iron related disorders.

Genes encoding small molecule transporters were differentially expressed, including carriers of glucose (SLC2A1, SLC5A4), chloride (SLC12A6), sodium (SLC13A3), monoamine (SLC18A2) and others (SLC26A4, SLC17A6). These transporters might help in exchange of nutrients and metabolites. Members of the cytochrome P and B family of proteins were also differentially expressed. Transcripts encoding VDP, RSAFD1, C1QG and OXA1L were significantly repressed by increasing DHA.

Based upon the above results, the present invention has shown that DHA and ARA can positively influence the transport and exchange of important nutrients and metabolites in the body. This may be important in biological processes ranging from nervous system function to muscle contraction to insulin release.

### G-Proteins and Signaling

Numerous genes encoding G-protein activity were differentially regulated. The majority of those were induced by high levels of DHA. For example, GNA13, GNA14, PTHR2, RCP9 and FZD3 showed increased expression in both DHA groups. EDG7, SH3TC2, GNRHR, ADRA1A, BLR1, GPR101, GPR20 and OR8G2 were downregulated in L/C and upregulated in L3/C.

DHA regulates G-protein signaling in the brain and retina. Salem, N., et al., Mechanisms of Action of Docosahexaenoic Acid in the Nervous System, Lipids 36(9): 945-59 (2001). G-proteins are membrane-associated proteins which promote exchange of GTP for GDP and regulate signal transduction and membrane traffic. Bomsel, M., & Mostov, K., Role of Heterotrimeric G Proteins in Membrane Traffic, Mol. Biol. Cell. 36(9): 945-59 (2001). GNA13 deficiency impairs angiogenesis in mice while GNA14 activates the NF-κB signaling cascade. Offermanns, S., et al., Vascular System Defects and Impaired Cell Chemokinesis as a Result of Galpha13 Deficiency, Science 275(5299): 533-36 (1997); Liu, A. M. & Wong, Y. H., Activation of Nuclear Factor κB by Somatostatin Type 2 Receptor in Pancreatic Acinar AR42J Cells Involves Gα14 and Multiple Signaling Components: A Mechanism Requiring Protein Kinase C, Calmodiulin-Dependent Kinase II, ERK, and c-Src, J. Biol. Chem. 280(41): 34617-25 (2005). Parathyroid hormone receptor 2 (PTHR2) is activated by parathyroid hormone and is relatively abundant in the CNS. Usdin, T. B., et al., New Members of the Parathyroid Hormone/Parathyroid Hormone Receptor Family: the Parathyroid Hormone 2 Receptor and Tuberoinfundibular Peptide of 39 Residues, Front Neroendocrin. 21(4): 349-83 (2000); Harzenetter, M. D., et al., Regulation and Function of the CGRP Receptor Complex in Human Granulopoiesis, Exp. Hematol. 30(4): 306-12 (2002). RCP9, also known as calcitonin gene-related peptide-receptor component protein, may have a role during hematopoiesis.

Another gene modulated by DHA and ARA supplementation includes FZD3 (frizzled, drosophilia, homolog of, 3). The FZD3 array results were confirmed by SYBR green real time PCR assay. G-Proteins are involved in the signaling mechanism, which uses the exchange of GDP for GTP as a molecule “switch” to allow or inhibit biochemical reactions inside the cell. Members of the FZD family are receptors for secreted WNT glycoproteins, which are involved in developmental control. RT-PCR and quantitative TaqMan analysis detected wide expression of FZD3, with highest levels in the limbic areas of the CNS and significant levels in testis, kidney, and uterus, as well as in a neuroblastoma cell line. C. F. Sala, et al., Identification, Gene Structure, and Expression of Human Frizzled-3 (FZD3), Biochem. Biophys. Res. Commun. 273(1):27-34 (2000). Tissir and Goffinet showed expression of FZD3 during postnatal CNS development in mice. Tissir, F. & Goffinet, A. M., Expression of Planar Cell Polarity genes During Development of the Mouse CNS, Eur. J. Neurosci. 23(3): 597-607 (2006).

The frizzled 3 (FZD3) gene is located on chromosome 8p21, a region that has been implicated in schizophrenia in genetic linkage studies. A strong association has been shown between the FZD3 locus and schizophrenia in Chinese population. Y. Zhang, et al., Positive Association of the Human Frizzled 3 (FZD3) Gene Haplotype with Schizophrenia in Chinese Han Population. Am. J. Med. Genet. B. Neuropsychiatr. Genet. 129(1):16-9 (2004); J. Yang, et al., Association Study of the Human FZD3 Locus with Schizophrenia, Biol. Psychiatry 54(11):1298-301 (2003).

Frizzled 3 (FZD3) can be a candidate tumor suppressor gene as loss of heterozygosity at chromosome 8p21 is detected in human breast and ovarian cancers. FZD3 has also been proposed as an important gene implicated in the neurogenesis of the CNS during embryogenesis. H. Kirikoshi, et al., Molecular Cloning and Genomic Structure of Human Frizzled-3 at Chromosome 8p21 Biochem. Biophys. Res. Commun. 271(1):8-14 (2000). As shown in Table 4, FZD3 has been upregulated in baboon infants in the L and L3 groups via DHA and ARA supplementation. Thus, it is believed that DHA and ARA supplementation has a beneficial effect on the incidence of schizophrenia or tumor suppression, among other things.

Neuropeptide Y is a 36-amino acid peptide with strong orexigenic effects in vivo. Tatemoto, K., Neuropeptide Y: Complete Amino Acid Sequence of the Brain Peptide, Proc. Natl. Acad. Sci. 79(18): 5485-89 (1982). Two major subtypes of NPY (Y1 and Y2) have been defined by pharmacologic criteria. NPY1R was suggested to be unique for the control of feeding. Gehlert, D. R., Multiple Receptors for the Pancreatic Polypeptide (PP-fold) Family: Physiological Implications, Proc. Soc. Exp. Biol. Med. 218(1): 7-22 (1998). Pedrazzini, et al. observed a moderate but significant decrease in food intake in mice lacking the NPY1R gene. Pedrazzini, T., et al., Cardiovascular Response, Feeding Behavior and Locomotor Activity in Mice Lacking the NPY Y1 Receptor, Nat. Med. 4(6): 722-26 (1998). Leptin suppresses feeding and decreases adiposity in part by inhibiting hypothalamic Neuropeptide Y synthesis and secretion.

EDG7 (endothelial differentiation, lysophosphatidic acid G-protein-coupled receptor, 7) mediates calcium mobilization. Bandoh, K., et al., Molecular Cloning and Characterization of a Novel Human G-Protein-Coupled Receptor, EDG7, for Lysophosphatidic Acid, J. Biol. Chem. 274(39): 277776-85 (1999). Mutation in the SH3TC2 gene causes childhood-onset of a neurodegenerative disorder affecting motor and sensory neurons. Senderek, J., et al., Mutations in a Gene Encoding a Novel SH3/TPR Domain Protein Cause Autosomal Recessive Charcot-Marie-Tooth Type 4C Neuropathy, Am. J. Hum. Genet. 73(5):1106-19 (2003).

Several signaling proteins (NF1, WSB1, SOCS4, RIT1, CD8B1, OR2A9P and RERG) were upregulated in both groups. Genes that are upregulated in L3/C and downregulated in L/C were also observed. For example, PDE4D, KRAS, ITGA2, PLCXD3, WNT8A, ARHGAP4, RAPGEF6, OR2F1/OR2F2, CCM1 and SFRP2 were upregulated in L3/C and downregulated in L/C. Several genes (WNT10A, ADCY2, OGT, DDAH1 and BCL9) were upregulated in L/C and downregulated in L3/C. IQGAP3, GCGR, APLN, CYTL1, GRP, LPHN3, CNR1, VAV3 and MCF2 were downregulated in both the groups.

Another of the genes upregulated in the cerebral cortex by DHA and ARA supplementation was NF1. NF1 expression levels were confirmed by QRT-PCR. Neurofibromatosis type 1 (NF1) is a disorder characterized particularly by “café-au-lait” spots and fibromatous tumors of the skin with an incidence of approximately 1 in 3000 people worldwide. Half of all patients present osseous manifestations, such as congenial pseudarthrosis. T. Kuorilehto, et al., NF1 Gene Expression in Mouse Fracture Healing and in Experimental Rat Pseudarthrosis, J. Histochem. Cytochem. 54(3):363-370 (2005).

NF1 gene expression and function are required for normal fracture healing. Id. Individuals with germline mutations in NF1 are predisposed to the development of benign and malignant tumors of the peripheral and central nervous system. Y. Zhu, et al., Inactivation of NF1 in CNS Causes Increased Glial Progenitor Proliferation and Optic Glioma Formation. Development. 132(24):5577-88 (2005). Loss of neurofibromin expression have been observed in a variety of NF1-associated tumors, including astrocytomas. D. H. Gutmann, et al., Loss of Neurofibromatosis 1 (NF1) Gene Expression in NF1-Associated Pilocytic Astrocytomas, Neuropathol. Appl. Neurobiol. 26:361-367 (2002); L. Kluwe, et al., Loss of NF1 Alleles Distinguish Sporadic from NF1-Associated Pilocytic Astrocytomas, J. Neuropathol. Exp. Neurol. 60:917-920 (2001).

In the L group, the NF1 gene was upregulated by only 2%, but in the L3 group, the gene was upregulated 27%, as compared to the control group. It is believed, therefore, that the upregulation of NF1 by DHA and ARA supplementation in infancy can prevent the later development of various tumors.

WSB1 is a SOCS-box-containing WD-40 protein expressed during embryonic development in chicken. Vasiliauskas, D. S., et al., SwiP-1: Novel SOCS Box Containing WD-Protein Regulated by Signalling Centres and by Shh During Development, Mech. Dev. 82(1-2):79-94 (1999). RAS and RAS related gene families of small GTPases (RIT1, KRAS, RERG and RAPGEF6) were upregulated by increasing DHA.

Diets deficient in n-3 PUFA induce substitution of n-6 DPA (22:5n-6) in neural membranes, and impairment of functions mediated by G protein mediated signaling, such as visual perception, learning and memory, and olfactory discrimination. Evidence indicates that this results in reduced rhodopsin activation, and signaling in rod outer segments compared to DHA-replete animals.

The results of the invention have illustrated that DHA and ARA supplementation may positively affect the signaling of G-proteins by allowing them to properly regulate cell processes. A malfunction in G-protein signaling may lead to diseases or disorders such as schizophrenia, tumors, or overweight. Thus, supplementation with DHA and ARA may aid in preventing or treating schizophrenia or tumors, may suppress appetite, and may aid in fracture healing.

### Development

Table 13 shows differential expression of 24 genes related to development.

TABLE 13
Development gene modulation in expression profiles.

| Development | Gene Symbol | Unigene ID | L1 | L3 |
|---|---|---|---|---|
| Nervous system | TIMM8A | Hs.447877 | 1.04 | 1.57 |
| | NRG1 | Hs.453951 | 1.02 | 1.21 |
| | SEMA3D | Hs.201340 | 1.10 | 1.14 |
| | NUMB | Hs.585653 | 1.01 | 1.10 |
| | HES1 | Hs.250666 | −1.30 | −1.63 |
| | SIM1 | Hs.520293 | −1.16 | −1.16 |
| | GDF11 | Hs.591023 | −1.18 | 1.09 |
| | SMA3///SMA5 | Hs.482414/484969/ | −1.08 | 1.06 |
| | | 588240 | | |
| | SH3GL3 | Hs.270055/458285 | −1.16 | 1.04 |
| | FGF5 | Hs.37055 | 1.08 | −1.20 |
| | FGF14 | Hs.591206 | 1.01 | −1.10 |
| Muscle | C6orf97 | Hs.130239 | −1.03 | 1.34 |
| | CALD1 | Hs.490203 | 1.09 | 1.14 |
| Skeletal | BAPX1 | Hs.105941 | 1.05 | 1.08 |
| Heart | GATA4 | Hs.243987 | −1.02 | 1.22 |
| Epidermis | S100A7 | Hs.112408 | −1.06 | 1.27 |
| | FGF7 | Hs.122006 | 1.14 | 1.02 |
| | SCEL | Hs.115166 | −1.01 | −1.13 |
| Ectoderm/ | SMURF1 | Hs.189329 | 1.15 | 1.32 |
| Mesoderm | TCF21 | Hs.78061 | −1.12 | −1.18 |
| Gametogenesis | OTEX | Hs.196956 | 1.09 | 1.24 |
| | TCP11 | Hs.435371 | −1.02 | 1.08 |
| | CDV1 | Hs.528382 | −1.001 | −1.10 |
| | SPAG6 | Hs.527698 | −1.03 | −1.22 |

The products of 11 transcripts play a role in nervous system development. The expression of TIMM8A, NRG1, SEMA3D and NUMB genes were upregulated in both L/C and L3/C groups. HES1 and SIM1 were downregulated in both the groups. GDF11, SMA3/SMA5, SH3GL3 were downregulated in L/C and upregulated in L3/C. The mRNA levels of growth factors FGF5 and FGF14 displayed increased abundance in L/C and decreased abundance in L3/C.

TIMM8A, also known as Deafness/Dystonia Peptide 1 (DDP1), is a well conserved protein organized in mitochondrial intermembrane space. It belongs to a family of evolutionary conserved proteins that are organized in the mitochondrial intermembrane space. These proteins mediate the import and intersection of hydrophobic membrane proteins into the mitochondrial inner membrane. It is a homolog of yeast translocase of the inner mitochondrial membrane 8.

Loss of function in the TIMM8A gene causes Mohr-Tranebjaerg syndrome, a progressive neurodegenerative disorder resulting in deafness, blindness, dystonia and mental deficiency. Loss of function in the TIMM8A gene can also cause Jensen syndrome, a disorder which results in optocoacoustic nerve atrophy with dementia. L. Tranebjaerg, et al., A De Novo Missense Mutation in a Critical Domain of the X-linked DDP Gene Causes the Typical Deafness-Dystonia-Optic Atrophy Syndrome. Eur J Hum Genet. 8(6):464-67 (2000); S. Hofmann, et al., The C66W Mutation in the Deafness Dystonia Peptide 1 (DDP1) Affects the Formation of Functional DDP1 TIM13 Complexes in the Mitochondrial Intermembrane Space, J. Biol. Chem. 277(26):23287-93 (2002); L. Tranebjaerg, et al., Neuronal Cell Death in the Visual Cortex is a Prominent Feature of the X-linked Recessive Mitochondrial Deafness-Dystonia Syndrome Caused by Mutations in the TIMM8a Gene, Ophthalmic Genet. 22(4):207-23 (2001).

In the present study, TIMM8A was upregulated in the cerebral cortex. Specifically, it was upregulated by 4% in the L group and 57% in the L3 group as compared to the control group. TaqMan assay confirmed the array results. Thus, it is believed that upregulation of the TIMM8A gene through DHA and ARA supplementation in infancy can prevent the later onset of Mohr-Tranebjaerg syndrome, Jensen syndrome and other neurodegenerative disorders.

TIMM23, which is also known as TIM23, is a mitochondrial inner membrane protein and is essential for cell viability. Lohret T A, et al., Tim23, a Protein Import Component of the Mitochondrial Inner Membrane, is Required for Normal Activity of the Multiple Conductance Channel, MCC, J. Cell. Biol. 21; 137(2):377-86 (1997). TIM23 mRNA content per cell clearly increases during the late stage of pregnancy and the mammary gland function is activated at this stage and may trigger lactogenesis. Sun Y, et al., Hormonal Regulation of Mitochondrial Tim23 Gene Expression in the Mouse Mammary Gland, Mol. Cell. Endocrinol. 172(1-2):177-84 (2001). Impaired biogenesis of the human TIMM23 complex causing severe pleiotropic mitochondrial dysfunction may be involved in the neurodegenerative disease Mohr-Tranebjaerg syndrome. Rothbauer, U., et al., Role of the Deafness Dystonia Peptide 1 (DDP1) in Import of Human Tim23 into the Inner Membrane of Mitochondria, J. Biol. Chem. 276(40):37327-34 (2001).

Thus, because TIMM23 was upregulated in infant baboon thymus tissue and TIMM23 is involved in Mohr-Tranebjaerg syndrome, it is believed that DHA and ARA supplementation can prevent and/or treat Mohr-Tranebjaerg syndrome.

NRG1 is essential for the development and function of the CNS facilitating the neuronal migration and axon guidance. Bernstein, H. G., et al., Localization of Neuregulin-1Alpha (Heregulin-Alpha) and One of its Receptors, ErbB-4 Tyrosine Kinase, in Developing and Adult Human Brain, Brain Res. Bull. 69(5): 546-59 (2006). NUMB negatively regulates notch signaling and plays a role in retinal neurogenesis, influencing the proliferation and differentiation of retinal progenitors and maturation of postmitotic neurons. Dooley, C. M., et al., Involvement of Numb in Vertebrate Retinal Development: Evidence for Multiple Roles of Numb in Neural Differentiation in the Central-Nervous-System, J. Neuro. 54(2): 313-325 (2003). HES1 (Hairy/Enhancer of Split, Drosophila, Homolog of, 1), a basic helix-loop-helix protein, was downregulated. Decreased expression of HES1 is observed as neurogenesis proceeds; in case of persistent expression, differentiation of neuronal cells are blocked in the CNS. Ishibashi, M., et al., Persistent Expression of Helix-Loop-Helix Factor Hes-1 Prevents Mammalian Neural Differentiation in the Central-Nervous-System, Embo. J. 13(8): 1799-1805 (1994).

In an embodiment, therefore, the invention is directed to a method for regulating the development of a subject comprising administering to that subject a therapeutically effective amount of DHA and ARA. These LCPUFAs may be effective in preventing various neurodegenerative disorders via their ability to modulate development-related genes.

### Visual Perception

Nine transcripts having a role in visual perception were differentially expressed (Table 14).

TABLE 14
Visual perception gene modulation in expression profiles

| Gene Product | Unigene ID | L | L3 |
|---|---|---|---|
| Lumican (LUM) | Hs.406475 | 1.03 | 1.30 |
| Interphotoreceptor matrix proteoglycan 1 | Hs.590893 | −1.03 | 1.18 |
| (IMPG1) | | | |
| Echinoderm microtubule associated | Hs.24178 | 1.07 | 1.15 |
| protein like 2 (EML2) | | | |
| TIMP metallopeptidase inhibitor 3 (TIMP3) | Hs.297324 | 1.28 | 1.05 |
| Tetratricopeptide repeat domain 8 (TTC8) | Hs.303055 | 1.10 | 1.01 |
| IMP (inosine monophosphate) | Hs.534808 | −1.20 | −1.12 |
| dehydrogenase 1 (IMPDH1) | | | |
| Tubby like protein 2 (TULP2) | Hs.104636 | 1.07 | −1.15 |
| Retina and anterior neural fold homeobox | Hs.278957 | −1.10 | −1.24 |
| (RAX) | | | |
| Regulator of G-protein signalling 16 | Hs.413297 | 1.01 | −1.26 |
| (RGS16) | | | |

Genes coding for LUM, EML2, TIMP3 and TTC8 were overexpressed in both the supplemental groups. TaqMan assay showed a 5-fold greater upregulation of LUM than that shown in the microarray data. IMPG1 was upregulated in L3/C and downregulated in L/C. RGS16 and TULP2 were upregulated in L/C and downregulated in L3/C. RAX and IMPDH1 were downregulated in both the supplemental groups.

Lumican (LUM), a member of the small-leucine-rich-proteoglycan (SLRP) family, is an extracellular matrix glycoprotein widely distributed in mammalian connective tissues. E. C. Carlson, et al., Keratocan, a Cornea-specific Keratan Sulfate Proteoglycan, Is Regulated by Lumican, J. Biol. Chem. 280(27): 25541-47 (2005). It is present in large quantities in the corneal stroma and in interstitial collagenous matrices of the heart, aorta, skeletal muscle, skin, and intervertebral discs. S. Chakravarti & T. Magnuson, Localization of Mouse Lumican (Keratan Sulfate Proteoglycan) to Distal Chromosome 10, Mamm. Genome. 6(5):367-68 (1995). Lumican helps in the establishment of corneal stromal matrix organization during neonatal development in mice. Those lacking lumican exhibit several corneal related defects. Beecher, N., et al., NeoNatal Development of the Corneal Stroma in Wild-Type and Lumican-Null Mice, Invet. Opthalmol. Vis. Sci. 42(8): 1750-1756 (2006). It is important for corneal transparency in mice. Mutations in TIMP3 gene result in autosomal dominant disorder, Sorsby's fundus dystrophy, an age-related macular degeneration of retina. L1, Z., et al., TIMP3 Mutation in Sorsby's Fundus Dystrophy: Molecular Insights, Expert Rev. Mol. Med. 7(24) 1-15 (2005). It has been suggested that a possible mechanism for retinal degeneration in Sorsby's fundus dystrophy was traceable to nutrition. Clarke, M., et al., Clinical Features of a Novel TIMP-3 Mutation Causing Sorsby's Fundus Dystrophy: Implications for Disease Mechanism, Br. J. Opthamol. 85(12): 1429-1431 (2001).

Lumican-null mice exhibit altered collagen fibril organization and loss of corneal transparency. Carlson, et al., J. Biol. Chem. 280(27):25541-47. Lumican also significantly suppressed subcutaneous tumor formation in syngenic mice and induced and/or enhanced the apoptosis of these cells. Z. Naito, The Role of Small Leucine-rich Proteoglycan (SLRP) Family in Pathological Lesions and Cancer Cell Growth. J. Nippon. Med. Sch. 72(3):137-45 (2005). In breast cancer, decreased mRNA expression levels of Lumican are associated with rapid disease progression and a poor survival rate. Id. Lumican has been implicated as an apoptotic gene in breast, pancreatic and colorectal cancers. S. Troup, et al., Reduced Expression of the Small Leucine-rich Proteoglycans, Lumican, and Decorin Is Associated with Poor Outcome in Node-negative Invasive Breast Cancer, Clin. Cancer Res. 9(1):207-14 (2003); Y. P. Lu, et al., Lumican Expression in Alpha Cells of Islets in Pancreas and Pancreatic Cancer Cells, J. Pathol. 196(3):324-30 (2002); Y. P. Lu, et al., Expression of Lumican in Human Colorectal Cancer Cells, Pathol. Int. 52(8):519-26 (2002).

LUM was upregulated in both the L and L3 group in brain tissue. Thus, DHA and ARA supplementation has a beneficial effect in upregulating LUM expression and it is believed that such upregulation can slow disease progression and provide a higher survival rate among individuals with breast, pancreatic, or colorectal cancers. It is believed that DHA and ARA supplementation also aids in tumor suppression.

IMPG1 is a proteoglycan which participates in retinal adhesion and photoreceptor survival. Kuehn, M. H. & Hageman, G. S., Expression and Characterization of the IPM 150 Gene (IMPG1) Product, A Novel Human Photoreceptor Cell-Associated Chondroitin-Sulfate Proteoglycan, Matrix Bio. 18(5): 509-518 (1999). Higher amounts of DHA in the infant formula increased the expression of IMPG1. Expression of RAX transcript was decreased in both the supplemental groups. Increased RAX expression is seen in the retinal progenitor cells during the vertebrate eye development and is downregulated in the differentiated neurons. Mathers, P. H. & Jamrich, M., Regulation of Eye Formation by the Rx and Pax6 Homeobox Genes, Cell. Mol. Life. Sci. 57(2): 186-194 (2000); Furukawa, T., et al., Rax, Hes1 and Notch1 Promote the Formation of Muller Glia by Postnatal Retinal Progenitor Cells, Neuron. 26(2): 383-394 (2000). DHA is well known to promote neurite growth in the brain; this could be the possible reason for RAX downregulation in the present study.

Based upon the above results, DHA and ARA supplementation modulate genes which aid in preserving or developing visual heath. Supplementation may prevent or treat the development of visual diseases or disorders or may improve the development of visual components.

### Integral to Membrane/Membrane Fraction

Transcripts that are integral parts of biological membranes or within the membrane fractions were differentially expressed in the present invention. For example, EVER1, PERP, Cep192, SSFA2, LPAL2, TMEM20, TM6SF1 were upregulated in both the groups. ORMDL3, SEZ6L, HYDIN, TA-LRRP, PKDIL1 were upregulated in L3/C and downregulated in L/C. MFAP3L was upregulated in L/C and downregulated in L3/C. Transcripts of GP2 and SYNGR2 were downregulated in both the groups.

Numbers of transcripts were upregulated by increased DHA in the formulas. LCPUFA supplementation can affect biological membrane functions by influencing membrane composition and permeability, interactions with membrane proteins, membrane-bound receptor functions, photoreceptor signal transduction, and/or transport. Liefert, W. R., et al., Membrane Fluidity Changes are Associated with the Antiarrhythmic Effects of Docosahexaenoic Acid in Adult Rat Cardiomyocytes, J. Nutr. Biochem. 11(1): 38-44 (2000); Stillwell, W. & Wassail, S. R., Docosahexaenoic Acid: Membrane Properties of a Unite Fatty Acid, Chem. Phys. Lipids 126(1): 1-27 (2003); SanGiovanni, J. P. & Chew, E. Y., The Role of Omega-3 Long-Chain Polyunsaturated Fatty Acids in Health and Disease of the Retina, Prog. Retinal Eye Res. 24(1): 87-138 (2005). Mutations in EVER1 or transmembrane channel-like 6 (TMC6) gene cause epidermodysplasia verruciformis, a type of skin disorder. Ramoz, N., et al., Mutations in Two Adjacent Novel genes are Associated with Epidermodysplasia Verruciformis, Nat. Genet. 32(4): 579-81 (2002). HYDIN is a novel gene and nearly-complete loss of its function due to mutations causes congenital hydrocephalus in mice. Davy, B. E. & Robinson, M. L., Congenital Hydrocephalus in Hy3 Mice is Caused by a Frameshift Mutation in Hydin, a Large Novel Gene, Hum. Mol. Gen. 12(10): 1163-1170 (2003). The exact function of GP2 is unknown, but it has been associated with the secretory granules in the pancreas. Yu, S., et al., Effects of GP2 Expression on Secretion and Endocytosis in Pancreatic AR4-2J Cells, Biochem. & Biophys. Res. Comm. 322(1): 320-325 (2004).

PERP (p53 Effector Related to PMP22) was expressed in the brain via DHA and ARA supplementation. PERP is a putative transmembrane receptor and a tumor suppressor gene. PERP knockout mice die after birth due to compromised adhesion and dramatic blistering in stratified epithelia. Loss of PERP might be associated with ectodermal dysplasia syndromes or an enhanced spontaneous risk of cancer by impairing the tumor suppression activity of both the p53 and p63 pathways. During normal zebrafish development, PERP is required for the survival of notochord and skin cells.

Thus, DHA and ARA supplementation may affect membrane/membrane functions by influencing (1) membrane composition and permeability, (2) interactions with membrane proteins, (3) membrane-bound receptor functions, (4) photoreceptor signal transduction, and/or (5) transport.

### Programmed Cell Death/Apoptosis

Transcripts with apoptotic activity were differentially expressed. Seven out of nine transcripts in the present study were upregulated with increasing DHA, including CARD6, TIA1, BNIP1, FAF1, GULP1, CASP9 and FLJ13491. Programmed cell death (PCD) plays an important role during the development of immune and nervous systems. Kuida, K., et al., Decreased Apoptosis in the Brain and Premature Letharlity in CPP32-Deficient Mice, Nature 384(6607): 368-372 (1996). Jacobson, et al. proposed PCD as an important event in eliminating unwanted cells during development. Mice with targeted deletion of CASP3 die perinatally due to vast excesses of cells deposition in their CNS as a result of decreased apoptotic activity. Jacobson, M. D., et al., Programmed Cell Death in Animal Development, Cell 88(3): 347-354 (1997). CARD6 (caspase recruitment domain protein 6) was upregulated in both the groups. It is a microtubule-interacting protein that activates NF-KB and takes part in the signaling events leading to apoptosis. Dufner, A. S., et al., Caspase Recruitment Domain Protein 6 is a Microtubule-interacting Protein that Positively Modulates NF-KB Activation, Proc. Natl. Acad. Sci. 103(4): 988-93 (2006). TIA1 was upregulated in L3/C and downregulated in L/C in the present invention. TIA1 is a member of RNA-binding protein family with pro-apoptotic activity, and it silences the translation of cyclooxygenase-2 (COX2). Narayanan, et al. suggested that DHA indirectly increases the expression of genes which downregulate COX2 expression. Narayanan, B. A., et al., Docosahexaenoic Acid Regulated Genes and Transcription Factors Inducing Apoptosis in Human Colon Cancer Cells, Int. J. Oncol. 19(6): 1255-62 (2001). The COX2 enzyme catalyzes the rate-limiting step for prostaglandin production, which influence many processes including inflammation. Dixon, D. A., et al., Regulation of Cyclooxygenase-2 Expression by the Translational Silencer TIA-1, J. Exp. Med. 198(3): 475-481 (2003). Downregulation of TIA1 in L/C could be due to the influence of ARA, the major COX2 substrate, rather than that of DHA, which is a competitive inhibitor. GULP1 assists in efficient removal of the apoptotic cells by phagocytosis. Su, H. P., et al., Interaction of CED-6/GULP, an Adapter Protein Involved in Englufinent of Apoptotic Cells with CED-1 and CD91/Low Density Lipoprotein Receptor-Related Protein (LRP), J. Bio. Chem. 277(14): 11772-11779 (2002). CASP9 activates caspase activation cascade and is an important component of mitochondrial apoptotic pathway. Brady, et al., Regulation of Caspase 9 Through Phosphorylation by Protein Kinase C Zeta in Response to Hyperosmotic Stress, Mol. Cell. Bio. 25(23): 10543-55 (2005).

The results discussed above indicate that the modulation of these genes may assist in the elimination of unwanted cells as a part of programmed cell death or apoptosis. This result is important in the development of a healthy immune and nervous system. The modulation caused by DHA and ARA supplementation may also be useful in preventing or treating inflammation in a subject.

### Cytoskeleton and Cell Adhesion

In the present invention, dietary LCPUFAs regulated expression of a number of transcripts involved in cytoskeleton and cell adhesion. In fact, the expression of 27 ps involved in cytoskeleton was altered. Genes encoding Myosin isoforms MYO1A, MYO5A and MYO1E were changed. MYO1A and MYO5A were upregulated with increasing amounts of DHA whereas MYO1E showed decreased expression. Myosin-1 isoforms are membrane associated molecular motors which play essential roles in membrane dynamics, cytoskeletal structure and signal transduction. Sokac, et al., Regulation and Expression of Metazoan Unconventional Myosins, in International Review of Cytology—A Survey of Cell Biology, Vo. 200: 197-304 (2000).

Expression of Collagen types IV and IX were altered by dietary LCPUFA. COL4A6 and COL9A3 showed increased expression whereas COL4A2 and COL9A2 showed decreased expression with increasing DHA. Type IV collagen is the major component of the basement membrane. Mild forms of AIport nephropathy are associated with deletion in COL4A6 gene and eye abnormalities are common in people afflicted with AIport syndrome. Mothes, et al., AIport Syndrome Associated with Diffuse Leiomyomatosis: COL4A5-COL4A6 Delection Associated with a Mild Form of AIport Nephrophathy, Nephrol. Dial. Transplant, 17(1): 70-74 (2002); Colville, et al., Ocular Manifestation of Autosomal Recessive AIport Syndrome, Ophtalmic Gen. 18(3): 119-128 (1997). Loss of the COL4A6 in epithelial basement membrane occurs in the early stage of cancer invasion. The expression of the COL4A6 was down-regulated in colorectal cancer. Leiomyomata of the esophagus is also associated with deletion in COL4A6 gene.

WASL, also known as neural WASP (WASP), was upregulated in both the groups. Actin cytoskeleton regulation is vital for brain development and function. WASL is an actin-regulating protein and mediates filopodium formation. Miki, et al., Induction of Filopodium Formation by a WASP Subcellular Localization and Function, Nature 391(6662): 93-96 (1998); Wu, et al., Focal Adhesion Kinase Regulation of N-WASP Subcellular Localization and Function, J. Bio. Chem. 279(10): 9565-76 (2004); Suetsugu, et al., Regulation of Actin Cytoskeleton by mDab1 through N-WASP and Ubiquitination of mDab1, Biochem. J. 384: 1-8 (2004). HIP1 (huntingtin interacting protein 1) and HOOK2 (hook homolog 2) were downregulated in both the groups.

The expression levels of 15 transcripts involved in cell adhesion changed as a result of dietary LCPUFA. For example, BTBD9, CD44, ARMC4, CD58, LOC389722 and PCDHB13 showed increased expression in both the groups. Glycoprotein CD44 is a cell-surface adhesion molecule that is involved in cell-cell and cell-matrix interactions while PCDHB13 is a member of protocadherin beta family of transmembrane glycoproteins. Wu, et al., A Striking Organization of a Large Family of Human Neural Cadherin-like Cell Adhesion Genes, Cell 97(5) 779-790 (1999). NLGN3 and CYR61 were downregulated in both groups.

The proper function of cytoskeletal and cell adhesion is important for the normal functioning of living organisms. Cell adhesion proteins hold together the components of solid tissues. They are also important for the function of migratory cells like white blood cells. Certain cancers involve mutations in genes for adhesion proteins that result in abnormal cell-to-cell interactions and tumor growth. Cell adhesion proteins also hold synapses together, which may affect learning and memory. In Alzheimer's disease there is abnormal regulation of synaptic cell adhesion. The results have shown that DHA and ARA can modulate genes involved with proper cytoskeletal and cell adhesion. Thus, a method of the present invention involves supplementing a subject with DHA and ARA in order to treat or prevent cancer or Alzheimer's disease, improve memory, or allow the migration of white blood cells.

### Peptidases

Several transcripts having peptidase activity were differentially expressed. SERPINB6 was significantly upregulated in L3/C and downregulated in L/C. Of note, the ADAM families of proteins (ADAM17, ADAM33, ADAM8, and ADAMTS16) were upregulated and ADAMTS15 was downregulated in both the supplemental groups. ADAM proteins are membrane-anchored glycoproteins named for two of the motifs they carry: an adhesive domain (disintegrin) and a degradative domain (metalloprotease). These proteins are involved in several biological processes including cell-cell interactions, heart development, neurogenesis and muscle development. ADAM17 is required for proteolytic processing of other proteins and has been reported to participate in the cleaving of the amyloid precursor protein. Loss of ADAM17 is reported in abnormalities associated with heart, skin, lung and intestines. Real time PCR confirmed the array results of ADAM17.

ADAM17 is also known as Tumor Necrosis Factor-Alpha Converting Enzyme (TACE). ADAM17 plays a neuroprotective role by cleaving of the amyloid precursor protein (APP) within the amyloid-beta (Aβ) sequence and thus play a key role in Alzheimer's disease process by preventing the formation of toxic amyloid-beta peptides. Buxbaum J D, et al., Evidence that Tumor Necrosis Factor Alpha Converting Enzyme is Involved in Regulated Alpha-Secretase Cleavage of the Alzheimer Amyloid Protein Precursor, J. Biol. Chem. 273:27765-27767 (1998); Endres K, et al., Shedding of the Amyloid Precursor Protein-Like Protein APLP2 by Disintegrin-Metalloproteinases, FEBS J. 272 (22):5808-5820 (2005). Additionally, aspirin induces platelet receptor shedding via ADAM17. Aktas B, et al., Aspirin Induces Platelet Receptor Shedding via ADAM17 (TACE), J. Biol. Chem. 280(48):39716-22 (2005).

A lack of ADAM17 leads to developmental abnormalities in mice, including defects in epithelial structures such as skin and intestines, as well as in morphogenesis of the lung. Peschon J J, et al., An Essential Role for Ectodomain Shedding in Mammalian Development, Science 282(5392):1281-4 (1998); Zhao J, et al., Pulmonary Hypoplasia in Mice Lacking Tumor Necrosis Factor-Alpha Converting Enzyme Indicates an Indispensable Role for Cell Surface Protein Shedding During Embryonic Lung Branching Morphogenesis. Dev. Biol. 232(1):204-18 (2001). Thus, it is believed that the upregulating effect of DHA and ARA on ADAM17 can prevent abnormalities in epithelial structures and heart development and can prevent or treat Alzheimer's.

ADAM17 mediates regulated ectodomain shedding of the severe-acute respiratory syndrome-coronavirus (SARS-CoV) Receptor, Angiotensin-converting enzyme-2 (ACE2). Lambert, D. W., et al., Tumor Necrosis Factor-Alpha Convertase (ADAM17) Mediates Regulated Ectodomain Shedding of the Severe-Acute Respiratory Syndrome-Coronavirus (SARS-CoV) Receptor, Angiotensin-Converting Enzyme-2 (ACE2). J. Biol. Chem. 280(34):30113-9 (2005). It has also been shown that mice lacking ADAM17 and ADAM19 have exacerbated defects in heart development. Horiuchi K, et al., Evaluation of the Contributions of ADAMs 9, 12, 15, 17, and 19 to Heart Development and Ectodomain Shedding of Neuregulins Beta1 and Beta2, Dev. Biol. 283(2):459-71 (2005). The heart abnormalities observed in mice lacking functional ADAM17 are thickened and misshapen semilunar valves (aortic and pulmonic valves) and atrioventricular valves. Jackson, L. F., et al., Defective Valvulogenesis in HB-EGF and TACE-Null Mice is Associated with Aberrant BMP Signaling, EMBO J. 22(11):2704-16 (2003).

ADAM33 is a member of the ‘disintegrin and metalloprotease domain’ family of proteins and has been recently implicated in asthma and bronchial hyperresponsiveness_by positional cloning. Van Eerdewegh, P., et al., Association of the ADAM33 Gene with Asthma and Bronchial Hyperresponsiveness, Nature 418:426-30 (2002).

ADAM33 occurs in smooth muscle bundles and around embryonic bronchi, strongly suggesting that it might play an important role in smooth muscle development and function. Haitchi H M, et al., ADAM33 Expression in Asthmatic Airways and Human Embryonic Lungs, Am. J. Respir. Crit. Care Med. 171(9):958-65 (2005). ADAM33 protein in both differentiated and undifferentiated embryonic mesenchymal cells suggests that it may be involved in airway wall “modeling” and may additionally be involved in determining lung function throughout life. Id.; Holgate, S T, et al., ADAM33: a Newly Identified Protease Involved in Airway Remodeling, Pulm. Pharmacol. Ther. 19(1):3-11 (2006). In murine models ADAM33 mRNA expression increases during embryonic lung development and remains into adulthood. Id. High-level expression in smooth muscles and fibroblasts suggest that ADAM33 plays a role in airway remodeling in asthmatics. Lee, J Y, et al., A Disintegrin and Metalloproteinase 33 Protein in Asthmatics: Relevance to Airflow Limitation, Am. J. Respir. Crit. Care Med. (Dec. 30, 2005).

Because ADAM33 was upregulated in both the L group and the L3 group of neonatal baboons, the inventors believe that DHA and ARA supplementation aids in airway wall “modeling” and smooth muscle development and function.

ADAM8 (a disintegrin and metalloproteinase domain 8) was expressed in the liver via DHA and ARA supplementation. ADAM8, also known as CD156, is highly expressed in monocytes, neutrophils, and eosinophils. It plays an important role in asthma disease. Recently, it was discovered that ADAM8 significantly inhibited experimentally induced asthma in mice. Thus, ADAM8 may also play a role in allergic diseases. ADAM8 plays a role in regulating monocyte adhesion and migration. Peroxisome proliferator-activated receptor-γ activation could also lead to increased expression of ADAM8.

CTSB (Cathepsin B), also known as amyloid precursor protein secretase (APPS), was upregulated. It is involved in the proteolytic processing of amyloid precursor protein. Felbor, et al. reported deficiency of CTSB results in brain atrophy and loss of nerve cells in mice. Felbor, et al., Neuronal Loss and Brain Atrophy in Mice Lacking Cathepsis V and L, Proc. Natl. Acad. Sci. 99(12) 7883-7888 (2002). CTSC (cathepsin C) was downregulated in the L/C group and upregulated in the L3/C group. Loss of function mutations in CTSC gene are associated with tooth and skin abnormalities. Toomes, et al., Loss-of-Function Mutations in the Cathepsin C Gene Result in Periodontal Disease and Palmoplantar Keratosis, Nat. Genet. 23(4): 421-424 (1999).

Cathepsin B (CTSB) was shown to be expressed in the brain due to DHA and ARA supplementation. Cathepsin B is also known as amyloid precursor protein secretase (APPS) and is involved in the proteolytic processing of amyloid precursor protein (APP). Incomplete proteolytic processing of APP has been suggested to be a causative factor in Alzheimer's disease. CTSB localization in placental and decidual macrophages suggests a role in the physiological function of these cells in mediating villous angiogenesis and decidual apoptosis. CTSB deficient mice show a reduction in premature intrapancreatic trypsinogen activation. It has been reported that combined deficiency of CTSB and CTSL results in neuronal loss and brain atrophy, suggesting that CTSB and CTSL are essential for maturation and integrity of the CNS.

NAALAD2 was upregulated while PAPLN, RNF130, TMPRSS2, PGC, CPZ, FURIN were downregulated. CPZ interacts with WNT proteins and may regulate embryonic development; however, its expression in adult tissues is less abundant. TPP2 and SPPL2B showed increased expression in L/C and decreased expression in L3/C. PAPPA, GZMA, SERPINA1, QPCTL transcripts were downregulated in L/C and upregulated in L3/C. Several hypothetical proteins (FLJ10504, FLJ30679, FLJ90661, FLJ25179, DKFZp686L1818) were differentially expressed.

Based upon the above results, the inventors have shown that DHA and ARA supplementation are effective in modulating peptidase genes. Accordingly, DHA and ARA are useful in prevention or treating abnormalities in the skin, heart, lung and/or intestines. As part of the method of the present invention, DHA and ARA may be especially useful in aiding the maturation and integrity of the lungs and/or CNS. DHA and ARA may also be useful in preventing or treating asthma or allergic disease.

### Cell Cycle, Cell Growth and Cell Proliferation

Fifteen transcripts having a role in cell cycle regulation, growth and proliferation were differentially expressed. Four of the transcripts SESN3, RAD1, GAS1 and PARD6B involved in cell cycle regulation were upregulated in both the groups.

SESN3 (sestrin 3) was expressed in the brain by DHA and ARA supplementation. Sestrins are cysteine sulfinyl reductases whose expression is modulated by p53. Budanov, et al., showed that sestrins are required for regeneration of peroxiredoxins which help in reestablishing the antioxidant properties. Budanov, et al., Regeneration of Peroxiredoxins by p53-Regulated Sestrins, Homologs of Bacterial AhpD, Sci. 304(5670): 596-600 (2004). The exact function of SESN3 is still not known.

Cell growth factors, INHBC and OGN were induced in both the groups. FGFR1OP is a positive regulator of cell proliferation and showed increased expression. KAZALD1, CDC20 and CDKN2C were down-regulated.

Growth arrest specific gene 1 (GAS1) expression is positively required for postnatal cerebellum development. Mice lacking GAS1 had significantly reduced cerebellar size compared to wild type mice. Liu, et al. proposed that GAS1 perform dual roles in cell cycle arrest and in proliferation in a cell autonomous manner. Liu, et al., Growth Arrest Specific Gene 1 is a Positive Growth Regulator for the Cerebellum, Dev. Biol. 236(1): 30-45 (2001). PARD6B has a role in axonogenesis. Brajenovic, et al., Comprehensive Proteomic Analysis of Human Par Protein Complexes Reveals an Interconnected Protein Network, J. Bio. Chem. 279(13): 12804-11 (2004).

INHBC is a member of transforming growth factor-beta (TGF-β) superfamily and is involved cell growth and differentiation. Osteoglycin (OGN) is also known as Mimecan and Osteoinductive factor (OIF). Mimecan is a member of small-leucine rich proteoglycan gene family and is a major component of cornea and other connective tissues. It has a role in bone formation, cornea development and regulation of collagen fibrillogenesis in corneal stroma. CDC20 regulates anaphase-promoting complex.

The inventors have shown in the present invention that DHA and ARA can modulate genes related to cell cycle, cell growth, and cell proliferation. As such, a method of the present invention comprises supplementing the diet of a subject with a therapeutically effective amount of DHA and ARA in order to enhance cell growth and proliferation and improve the cell cycle in general.

### Response to Stress

MSRA, SOD2, GSTA3 and GSR genes were differentially expressed. MSRA (peptide methionine sulfoxide reductase) was upregulated in both the supplemental groups. SOD2 was down-regulated in L/C and upregulated in L3/C. GSR was upregulated in the L/C and downregulated in the L3/C. GSTA3 was downregulated in both the groups.

Oxidative damage to proteins by reactive oxygen species is associated with oxidative stress, aging, and age-related diseases. MSRA is expressed in the retinal pigmented epithelial cells, neurons, and throughout the nervous system. Knock-outs of the MSRA gene in mice result in shortened life-spans both under normoxia and hyperoxia (100% oxygen) conditions. MSRA also participates in the regulation of proteins. MSRA plays an important role in neurodegenerative diseases like Alzheimer's and Parkinson's by reducing the effects of reactive oxygen species. Overexpression of MSRA protects human fibroblasts against H₂O₂-mediated oxidative stress.

Reactive oxygen species (ROS) can oxidize methoionine (Met) to methionine sulfoxide (MetO). The oxidized product, methinine sulfoxide, can be enzymatically reduced back to methionine by peptide methionine sulfoxide reductase. Overexpression of MSRA under elevated oxidative stress conditions predominantly in the nervous system markedly extended the life span of the Drosophilia. Methionine sulfoxide reductase is a regulator of antioxidant defense and life span in mammals.

SOD2 belongs to the iron/manganese superoxide dismutase family. It encodes a mitochondrial protein and helps in the elimination of reactive oxygen species generated within mitochondria. In the present study, increased amounts of DHA reduced the expression of glutathione-related proteins GSR and GSTA3.

The data in the present invention has shown that DHA and ARA supplementation are effective in modulating genes associated with stress response. Based upon these results, DHA and ARA supplementation are useful in preventing or treating oxidative stress, age-related disorders, and neurodegenerative diseases. In addition, DHA and ARA supplementation may aid in proper development and integrity of the retina, neurons, and nervous system. Supplementation of a therapeutically effective amount of DHA and ARA may also lengthen the life span of a subject.

### Kinases and Phosphatases

Phosphorylation and dephosphorylation of proteins control a multitude of cellular processes. Several proteins having kinase activity were altered in the present invention as a result of DHA and ARA supplementation. Of note, transcripts involving STK3, STK6, HINT3, TLK1, DRF1, GUCY2C and NEK1 were significantly upregulated with increasing DHA. A number of MAP kinases were downregulated in L3/C group, including MAP4K1, MAPK12, MAP3K2 and MAP3K3. Other transcripts which showed significantly decreased expression were CKM, LMTK2, NEK11, TNK1, BRD4 and MGC4796.

Transcripts having dephosphorylation activity, including ACPL2, KIAA1240, PPP2R3A, PPP1R12B, PTPRG, PPP3CA and ACPP were upregulated in L3/C group. MTMR2, PPP1R7, PTPRN2 and HDHD3 were significantly downregulated with increasing DHA.

### Transcription Factors

Several transcription factors are differentially expressed by dietary LCPUFA. Zinc finger proteins, Homeo box proteins and RNA Pol II transcription factors were among them. Several of the Zinc finger proteins were overexpressed in L3/C, which include ZNF611, ZNF584, ZNF81, ZNF273, ZNF547, MYNN, ZBTB11, PRDM7, JJAZ1, ZNF582, MLLT10, ZNF567, ZNF44, ZNF286, ZFX, NAB1, ZNF198, ZNF347 and ZNF207, while PCGF2, ZBTB9, ZNF297, WHSCIL1, SALL4, ZNF589, ZFY, ZNF146, ZNF419 and ZNF479 were repressed in L3/C group. Zinc finger proteins exhibit varied biological functions in eukaryotes including activation of transcription, protein folding, regulation of apoptosis, and lipid binding. Homeobox transcription factors, TGIF2, PHTF1, OTP and HHEX were induced whereas PHOX2A, IRX1 and MITF were repressed in L3/C. RNA Pol II transcription factors (BRCA1, TFCP2, CHD2, THRAP3, SMARCD2 and NFE2L2) showed increased expression in L3/C. However, transcripts for UTF1, POU2F2, ELL, POLR2C, THRAP5, TGIF and GLIS1 showed decreased expression in L3/C. SOX7 and SOX12, high mobility group (HMG) box proteins, were also differentially expressed. ZNF611 array expression results were confirmed by real time PCR.

BRCA1 is a tumor suppressor gene. BRCA1 was the first identified and cloned breast and ovarian cancer susceptibility gene. Miki Y., et al., A Strong Candidate for the Breast and Ovarian Cancer Susceptibility Gene BRCA1, Science 266(5182):66-71 (1994). Both hereditary and sporadic breast and ovarian tumors frequently have decreased BRCA1 expression. Wilcox C B, et al., High-Resolution Methylation Analysis of the BRCA1 Promoter in Ovarian Tumors, Cancer Genet. Cytogenet. 159(2):114-22 (2005). BRCA1 may contribute to its tumor suppressor activity, including roles in cell cycle checkpoints, transcription, protein ubiquitination, apoptosis, DNA repair and regulation of chromosome segregation. Venkitaraman A R. Cancer Susceptibility and the Functions of BRCA1 and BRCA2, Cell 108:171-182 (2002); Rosen E M, et al., BRCA1 Gene in Breast Cancer, J. Cell. Physiol. 196:19-41 (2003); Lou Z, et al., BRCA1 Participates in DNA Decatenation, Nat. Struct. Mol. Biol. 12:589-93 (2005); Zhang, J. & Powell, S. N., The Role of the BRCA1 Tumor Suppressor in DNA Double-Strand Break Repair. Mol. Cancer Res. 3(10):531-9 (2005).

The emerging picture is that BRCA1 plays an important role in maintaining genomic integrity by protecting cells from double-strand breaks (DSB) that arise during DNA replication or after DNA damage. Zhang & Powell, 2005. BRCA1 mutation carriers have a significantly increased risk of pancreatic, endometrial, and cervical cancers as well as prostatic cancers in men younger than age 65. Thompson, D. & Easton, D. F., Cancer Incidence in BRCA1 Mutation Carriers, J. Natl. Cancer Inst. 94:1358-1365 (2002).

BRCA1 was upregulated in both the L group and the L3 group, and, thus, it is believed that DHA and ARA supplementation lowers the risk of pancreatic, endometrial, cervical, and prostatic cancers and can suppress tumors.

### Receptor Activity

Transcripts performing receptor activities were differentially expressed. While increasing levels of DHA were associated with decreased expression of CD40, ITGB7, IL20RA, CD14, DOK3, MR1, BZRAP1, RARA, CD3D, IL1R1, MCP, and HOMER3 transcripts, increased expression was detected for FCGR2B, IL31RA, MRC2, SCUBE3, CR2, NCR2, CRLF2, SLAMF1, EGFR and KIR3DL2. Interestingly, retinoic acid receptor α (RARA) activity was decreased in both the groups. EGFR expression levels were confirmed by QRT-PCR.

### Ubiquitin Cycle

Twenty-five probe sets having a role in the ubiquitination process were differentially expressed. Interestingly, five members of F-box protein family (FBXL7, FBXL4, FBXL17, FBXW4 and FBXW8) showed increased expression in L3/C group. F-Box proteins participate in varied cellular processes such as signal transduction, development, regulation of transcription, and transition of cell cycle. They contain protein-protein interaction domains and participate in phosphorylation-dependent ubiquitination. Proteins associated with anaphase-promoting complex (CDC23 and ANAPC1) were downregulated in L3/C group.

### Others

Transcripts involved in 1) calcium ion binding (MGC33630, UMODL1, FLJ25818, S100Z, MGC12458, ITSN2 and PRRG3), 2) zinc ion binding (FGD5, ZFYVE28, PDLIM4, ZCCHC6, ZNF518 and INSM2), 3) ATP binding (MMAA and C6orf102), 4) GTP binding (DOCK5, DOCK6, DOCK10, MFN1 and GTP), 5) nucleic acid binding (IFIH1, C13orf10, DDX58, TNRC6C, RSN, ZCCHC5, DJ467N11.1, MGC24039 and LOC124245), 6) DNA binding (KIAA1305, HP1-BP74, H2AFY, C17orf31, HIST1H2BD and HIST1H1E) 7) protein binding (ABTB1, MGC50721, RANBP9, STXBP4, BTBD5 and KLHL14) and 8) protein folding (HSPB3, DNAJB12, FKBP11 and TBCC) were all differentially expressed. Also, several transcripts which play a role in RNA processing events were differentially expressed. For example, SFRS21P, LOC81691, EXOSC2, SFPQ, SNRPN and SFRS5 showed increased expression with increasing DHA whereas NOL5A, RBM19, NCBP2 and PHF5A showed decreased expression with increasing DHA. Transcripts related to immune response were also differentially expressed. For example, HLA-DPB1, MX2 and IGHG1 were overexpressed and PLUNC was underexpressed with increasing DHA.

A gene known as FOXP2 (Forkhead box P2), was upregulated in the cerebral cortex of baboons supplemented with DHA and ARA. In the L group, the gene was downregulated by 8%, but in the L3 group the gene was upregulated by 38%, as compared to the control group. FOXP2 is a putative transcription factor that plays an important role in neurological development. A mutation in FOXP2 can cause severe speech and language deficits. Recent studies in songbirds show that during times of song plasticity FOXP2 is upregulated in a striatal region essential for song learning. The gene has also been implicated in speech development. Therefore, the inventors believe that upregulation of FOXP2 through DHA and ARA supplementation aids neurological and speech development.

Other genes that were upregulated by DHA and ARA supplementation include XLC1 and 2. They are chemokines, C motif, ligands 1 & 2. Chemokines are a group of small (approximately 8 to 14 kD), mostly basic structurally related molecules that regulate cell trafficking of various types of leukocytes through interactions with a subset of 7-transmembrane G protein-coupled receptors. Chemokines also play fundamental roles in the development, homeostasis, and function of the immune system, and they have effects on cells of the central nervous system as well as on endothelial cells involved in angiogenesis or angiostasis. They are considered to be mediators of the immune response. Therefore, the inventors believe that upregulation of XLC1 or 2 via DHA and ARA supplementation improves function of the immune system.

Yet another gene that was upregulated by DHA and ARA supplementation was RNASE3. RNASE3, also known as Eosinophil Cationic protein, is a ribonuclease of the “A” family. It is localized to the granule matrix of the eosinophil and possess neurotoxic, helminthotoxic, and defense responses to bacteria and ribonucleolytic activities. It has been implicated in connection with cellular immunity. It is believed, therefore, that the upregulation of RNASE3 via DHA and ARA supplementation improves the function of the immune system.

NRF1 is a transcription factor that acts on nuclear genes encoding respiratory subunits and components of the mitochondrial transcription and replication machinery. NRF1 is well known to regulate mitochondrial DNA transcription and replication in various tissues. Knocking out the NRF1 gene leads to embryonic death around the time of the implantation in a mouse. May-Panloup P., et al., Increase of Mitochondrial DNA Content and Transcripts in Early Bovine Embryogenesis Associated with Upregulation of mtTFA and NRF1 Transcription Factors, Reprod. Biol. Endocrinol. 3:65 (2005).

It has been shown that NRF1 expression is down-regulated in the skeletal muscle of diabetic and prediabetic insulin-resistant individual. Patti, M. E., et al., Coordinated Reduction of Genes of Oxidative Metabolism in Humans with Insulin Resistance and Diabetes: Potential Role of PGC1 and NRF1, Proc. Natl. Acad. Sci. 100(14):8466-71 (2003). It has also been shown that NRF1 has a protective function against oxidative stress and that mice with somatic inactivation of NRF1 in the liver developed hepatic cancer. Parola, M. & Novo, E., Nrf1 Gene Expression in the Liver: a Single Gene Linking Oxidative Stress to NAFLD, NASH and Hepatic Tumours, J. Hepatol. 43(6):1096-7 (2005).

Intake of EPA and DHA increase the expression of NRF1. Flachs P, et al., Polyunsaturated Fatty Acids of Marine Origin Upregulate Mitochondrial Biogenesis and Induce Beta-Oxidation in White Fat, Diabetologia. 48(11):2365-75 (2005). It has also been suggested that NRF1 plays an important role in neuronal survival after acute brain injury. Hertel M, et al., Upregulation and Activation of the Nrf-1 Transcription Factor in the Lesioned Hippocampus, Eur. J. Neurosci. 15(10):1707-11 (2002).

Over-expression of NRF1 increases the intracellular glutathione level. Gamma-glutamylcysteinylglycine or glutathione (GSH) performs important protective functions in the cell through maintenance of the intracellular redox balance and elimination of xenobiotics and free radicals. Myhrstad M C, et al., TCF11/NRF1 Overexpression Increases the Intracellular Glutathione Level and Can Transactivate the Gamma-Glutamylcysteine Synthetase (GCS) Heavy Subunit Promoter, Biochim. Biophys. Acta. 1517(2):212-9 (2001).

It is believed that the upregulation of NRF1 through DHA and ARA supplementation in the present invention can be a method for improving brain development, health, and function.

STK3 is a gene which is also known as Mammalian Sterile 20-Like 2 (MST2) or Kinase Responsive to Stress 1 (KRS1). It is a member of the germinal center kinase group II (GCK II) family of mitogen-activated protein kinases. Dan I., et al., The Step 20 Group Kinases as Regulators of MAP Kinase Cascades, Trends Cell. Biol. 11:220-30 (2001). Emerging evidence suggests that the proapoptotic kinase MST2 acts in a novel tumor suppression pathway. O'Neill E E, et al., Mammalian Sterile 20-Like Kinases in Tumor Suppression: An Emerging Pathway, Cancer Res. 65(13):5485-7 (2005). Overexpression of MST2 induces apoptosis. O'Neill E, et al., Role of the Kinase MST2 in Suppression of Apoptosis by the Proto-Oncogene Product Raf-1, Science 306:2267-2270 (2004). STK3 was upregulated in both the L and L3 formula groups in the present study. Thus, it is believed that DHA and ARA supplementation is effective in tumor suppression via the upregulation of STK3.

RNASE3 is also known as Eosinophil cationic protein (ECP). It is a highly basic protein of the ribonuclease-A family that is released from matrix of eosinophil granules. RNASE3 possesses antiviral, antibactericidal, neurotoxic, helminthotoxic, and ribonucleolytic activities. Rosenberg, H. F., Recombinant Human Eosinophil Cationic Protein: Ribonuclease Activity is not Essential for Cytotoxicity, J. Biol. Chem. 270(14):7876-81 (1995); Kreuze, J. F., et al., Viral Class 1 RNase III Involved in Suppression of RNA Silencing, J. Virol. 79(11):7227-38 (2005). RNA silencing is a eukaryotic cellular surveillance mechanism that defends against viruses, controls transposable elements, and participates in the formation of silent chromatin. RNA silencing is also involved in post-transcriptional regulation of gene expression during developmental processes. RNASE3 enhances the suppression of RNA silencing. Kreuze, et al., 2005. It has also been shown that only human RNASE 3, among five human pancreatic-type RNASES, excels in binding to the cell surface and has a growth inhibition effect on several cancer cell lines. Maeda T, et al., RNase 3 (ECP) is an Extraordinarily Stable Protein Among Human Pancreatic-Type RNases, J. Biochem. 132(5):737-42 (2002).

RNASE2 is also known as Eosinophil-derived neurotoxin (EDN). It has been demonstrated that remarkable similarities exist between Eosinophil-derived neurotoxin and Eosinophil cationic protein. Hamann K J, et al., Structure and Chromosome Localization of the Human Eosinophil-Derived Neurotoxin and Eosinophil Cationic Protein Genes: Evidence for Intronless Coding Sequences in the Ribonuclease Gene Ssuperfamily, Genomics 7(4):535-46 (1990). EDN inactivates retroviruses in vitro. Rosenberg, H. F., Domachowske, J. B., Eosinophils, Eosinophil Ribonucleases, and their Role in Host Defense Against Respiratory Virus Pathogens, J. Leukoc. Biol. 70(5):691-8 (2001). EDN possesses antiviral, antibactericidal, cytotoxic, neurotoxic, helminthotoxic, dendritic cell chemotactic activities, and ribonucleolytic activities. Id.; Yang D, et al., Eosinophil-Derived Neurotoxin (EDN), an Antimicrobial Protein with Chemotactic Activities for Dendritic Cells, Blood 102(9):3396-403 (2003). EDN has also been shown to be responsible in part for the HIV-1 inhibitory activities in the supernatant of allogeneic mixed lymphocyte reaction. Rugeles M T, et al. Ribonuclease is Partly Responsible for the HIV-1 Inhibitory Effect Activated by HLA Alloantigen Recognition, AIDS 17:481-486 (2003).

Both RNASE2 and RNASE3 were upregulated in the baboon thymus in the presence of either 1.00% DHA or 0.33% DHA and 0.67% ARA supplementation. Thus, the present invention has shown that DHA and ARA supplementation can be effective in providing antiviral, antibactericidal, neurotoxic, helminthotoxic, and ribonucleolytic properties, cytotoxic, and dendritic cell chemotactic activities via the upregulation of RNASE2 and RNASE3.

TNNC1, also known as Troponin C, Cardiac (TNC), was shown in the present invention to be expressed in the liver. Contractions in striated muscles are regulated by the calcium-ion-sensitive, multiprotein complex troponin and the fribrous protein tropomyosin. The first mutation of the TNNC1 gene was identified in a patient with hypertrophic cardiomyopathy. This mutation is associated with a reduction in calcium sensitivity. The amino acid substitution TNNC1 (G159D) is localized in a domain of the protein constitutively occupied by Ca²⁺. This may change the affinity for Ca²⁺ and, thereby, alter the ability of the troponin complex to regulate myocardial contractility. Idiopathic dilated cardiomyopathy (DCM) is the most common cause of heart failure and cardiac transplantation in the young. The condition is characterized by unexplained left ventricle dilation, impaired systolic function, and nonspecific histologic abnormalities dominated by myocardial fibrosis. Patients may experience severe disease complications including arrhythmia, thromboembolic events, and sudden death. It has been proposed that DCM mutations in the troponin complex may induce a profound reduction in force generation leading to impaired systolic function and cardiac dilation. In addition, it is possible that the myocardium of mutation carriers may be more susceptible to environmental influences such as viruses and toxic agents.

Thus, it is believed that an increased expression of TNNC1 via DHA and ARA supplementation may prevent or treat malfunctions, diseases, or disorders of the heart, such as arrhythmia, thromboembolic events, and even heart failure.

ASB1 (ankyrin repeat- and socs box-containing protein) has been shown to be expressed in the liver due to DHA and ARA supplementation. ASB1 belongs to the suppressor of cytokine signaling (SOCS) box protein superfamily. The ankyrin-repeats are compatible with a role in protein-protein interactions. It has been shown that mice lacking the ASB1 gene display a dimunition of spermatogenesis with less complete filling of seminiferous tubules. However, overexpression of ASB1 had no apparent effects. It is believed, then, that DHA and ARA supplementation according to the method of the present invention may modulate the expression of ASB1 and aid in the proper development and activity of the reproductive system.

Cathepsin D (CTSD) is a lysosomal aspartic proteinase that has been shown to be expressed in the liver in the present invention. It plays an important role in the degradation of proteins and in apoptotic processes induced by oxidative stress, cytokines, and aging. Reduced activity of CTSD has been found in congenital ovine neuronal ceroid lipofuscinosis (CONCL), a type of neurodegenerative disease. CONCL is caused by a point mutation in the CTSD gene and is characterized by small brain size, pronounced neuronal loss, reactive astrocytosis, and infiltration of macrophages. CTSD cleaves beta-amyloid precursor protein near the beta secretase sites. It has been shown CTSD may play an important role in processing mutant Huntingtin protein (mHtt) in Huntington's disease. The inactive form of CTSD in the retinal pigment epithelium (RPE) in a transgenic mice model showed RPE atrophy, photoreceptor outer segment (POS) shortening and loss and accelerated debris accumulation. It has been shown that decreased CTSD expression levels in renal cell cancer specimens is associated with increased likelihood for the development of metastatic disease. CTSD deficiencies cause massive neuronal death in the central nervous system and may be the cause for lysosomal storage, stroke and age-related neurodegenerative diseases including Alzheimer's. Thus, the method of the present invention is useful in modulating CTSD expression and preventing or treating neurodegenerative and/or metastatic diseases through DHA and ARA supplementation.

LMX1B (LIM Homeobox Transcription Factor 1, beta) was expressed in the thymus upon DHA and ARA supplementation. Loss of function mutations in LMX1B causes nail patella syndrome (NPS). NPS is an autosomal dominant disorder affecting development of the limbs, kidney, eyes and neurologic functions. Lmx1b may have a unique role in neuronal migration in the developing spinal cord. The diminished pain responses in NPS patients may be due to the inability of afferent sensory neurons to migrate. Lmx1b is required for the development of 5-hydroxytryptamine neurons in the central nervous system in mice. Dreyer, et al. showed expression of LMX1B during joint and tendon formation. Dreyer, et al., Lmx1b Expression During Joint and Tendon Formation: Localization and Evaluation of Potential Downstream Targets, Gene Exp. Patterns 4(4): 397-405 (2004). LMX1B regulates the expression of multiple podocyte genes critical for podocyte differentiation and function.

Supplementation with DHA and ARA according to the method of the invention has been shown to modulate LMX1B expression and thereby prevent or treat autosomal disorders. In addition, DHA and ARA supplementation aids in proper development of the limbs, kidney, eyes, neurological system, and spinal cord via LMX1B modulation.

BHMT (betaine-Homocysteine methyltransferase) was expressed in the liver upon DHA and ARA supplementation. BHMT is an important zinc metalloenzyme in the liver. The expression of BHMT is confined mainly to the liver and its expression is reduced in cases of liver cirrhosis and liver cancer. BHMT is expressed abundantly in the nuclear region of the monkey eye lens and is developmentally regulated. As BHMT is abundantly present in the eye lens, it can be considered as an enzyme crystallin. Hyperhomocysteinemia is considered to be a risk factor for a number of important diseases like kidney failure, cardiovascular disorders, stroke, neurodegenerative diseases (including Alzheimer's) and neural tube defects. BHMT catalyzes the transfer of methyl groups from betaine to homocysteine to form dimethylglycine and methionine and helps in reducing the levels of homocysteine. Therefore, the present invention is useful in modulating the expression of BHMT in the liver and thereby promoting healthy liver function.

PPARD (peroxisome proliferator-activated receptor-Δ) was expressed in the liver upon DHA and ARA supplementation. C18 unsaturated fatty acids are known to activate human and mouse PPARD. Syndrome X or metabolic syndrome is a collection of obesity related disorders. PPARs are transcription factors and are involved in the regulation of genes in response to fatty acids. PPARD knockout mice were observed to be metabolically less active and glucose intolerant, whereas receptor activation improved insulin sensitivity. This suggests that PPARD ameliorates hyperglycemia and could suggest a therapeutic approach to treat type II diabetes. PPARD plays beneficial roles in cardiovascular disorders by inhibiting the onset of oxidative stress-induced apoptosis in cardiomyoblasts. Ligand activation of PPARD can induce terminal differentiation of keratinocytes. Burdick, et al. reviewed the literature on PPARD and reported from several recent studies that ligand activation of PPARD can induce fatty acid catabolism in skeletal muscle and is associated with improved insulin sensitivity, attenuated weight gain and elevated HDL levels. Burdick, et al., The Role of Peroxisome Proliferator-Activated Receptor-Beta/Delta in Epithelial Cell Growth and Differentiation, Cell Signal 18(1): 9-20 (2006). This suggests that PPARD can be used as target for treating obesity, dyslipidemias and type-2 diabetes. Increased expression of PPARD is observed during first and third trimester of pregnancy, indicating an important role in placental function.

Therefore, DHA and ARA supplementation according to the method of the present invention can modulate PPARD expression, improving insulin sensitivity, improving glucose intolerance, improving hyperglycemia, and treating obesity, dyslipidemias and type-2 diabetes.

Other genes that were affected by DHA and ARA supplementation are listed in Tables 15 and 16, respectively.

TABLE 15
Cerebral Cortex Genes Affected by
DHA and ARA Supplementation.¹

| | | L group (% | L3 group (% |
|---|---|---|---|
| | | regulation | regulation |
| | | as compared | as compared |
| | | to control | to control |
| Gene | Biological Activity | group) | group) |
| TIMM8A | Homolog of yeast | 4 | 57 |
| | translocase of inner | | |
| | mitochondrial membrane 8 | | |
| NF1 | Neurofibromatosis, type 1 | 2 | 27 |
| ADAM17 | Distintegrin and | 37 | 50 |
| | metalloproteinase domain 17 | | |
| BRCA1 | Breast cancer 1 gene | 4 | 35 |
| LUM | Lumican | 3 | 30 |
| FOXP2 | Forkhead box P2 | −8 | 38 |
| SPTLC2 | Serine palmitoyltransferase, | 26 | 40 |
| | LC base subunit 2 | | |
| FTH1 | Ferritin heavy chain 1 | −8 | 37 |
| OSBP2 | Oxysterol-binding protein 2 | −16 | 35 |
| NSMAF | Neural sphingomyelinase | −4 | 31 |
| | activation-asso factor | | |
| PDE3A | Phophodiesterdase, 3A | 8 | 30 |
| | cgmp-inhibited | | |
| SOD | Superoxide dimutase 2 | −7 | 29 |
| ACADSB | Acyl-coa dehydrogenase, | −11 | 38 |
| | short/branched chain | | |
| SFTPB | Surfactant, pulmonary | 3 | 35 |
| | associated protein B | | |

¹Positive values indicate upregulation; negative values indicate downregulation.

TABLE 16
Thymus Genes Affected by DHA and ARA Supplementation.

| | | L group (% | L3 group (% |
|---|---|---|---|
| | | regulation | regulation |
| | | as compared | as compared |
| | | to control | to control |
| Gene | Biological Activity | group) | group) |
| TOB1 | Transducer of ERBB2, 1 | 30 | 110 |
| XCL1 & | Chemokine, C motif, ligands | 40 | 32 |
| XCL2 | 1 &2 | | |
| RNASE3 | Ribonuclease A family 3 | 60 | 43 |
| SULT1C1 | Sulfotransferase family 1C, | 35 | 35 |
| | member 1 | | |
| HSPCA | Heat-shock, 90 KD protein 1, | −2 | 25 |
| | alpha | | |
| CD44 | CD44 antigen | 37 | 30 |
| CD24 | CD24 antigen | 43 | 28 |
| OSBPL9 | Oxysterol-binding protein-like | 3 | 20 |
| | protein 9 | | |
| FCER1G | FC fragment of IGE, receptor | 44 | 23 |
| | subunit 1 | | |
| KIR2DS1 | Killer cell immunoglobin-like | 30 | 10 |
| | receptor, two domains, short | | |
| | cytoplasmic tail, 1 | | |

Finally, 406 transcripts with no known gene ontology functions were differentially expressed. Several of these transcripts were among the most differentially expressed, among these, H63, LOC283403, FLJ13611, PARP6, C6orf111, C10orf67, TTTY8, C11orf1 and PHAX were upregulated, whereas transcripts for CHRDL2, TSGA13, RP4-622L5, MGC5391, RNF126P1, FAM19A2 and NOB1P were repressed considerably.

### Ingenuity Network Analysis

The inventors explored relationships among sets of genes using Ingenuity Systems network analysis. Out of 1108 differentially expressed probe sets in the present data, 387 probe sets (34.93%) were found in the Ingenuity Pathway Analysis (IPA) knowledge database, and are labeled “focus” genes. Based on these focus genes, IPA generated 41 biological networks, which are shown in Table 17.

Table 17 is contained on the submitted compact disc and is hereby incorporated by reference in its entirety. The file containing Table 17 is identified as Greenville-#576000-v1-2_{—}9_{—}07_Non-Provisional_Table_{—}17_(19400_.XLS; Size 38 KB; Created Feb. 23, 2007.

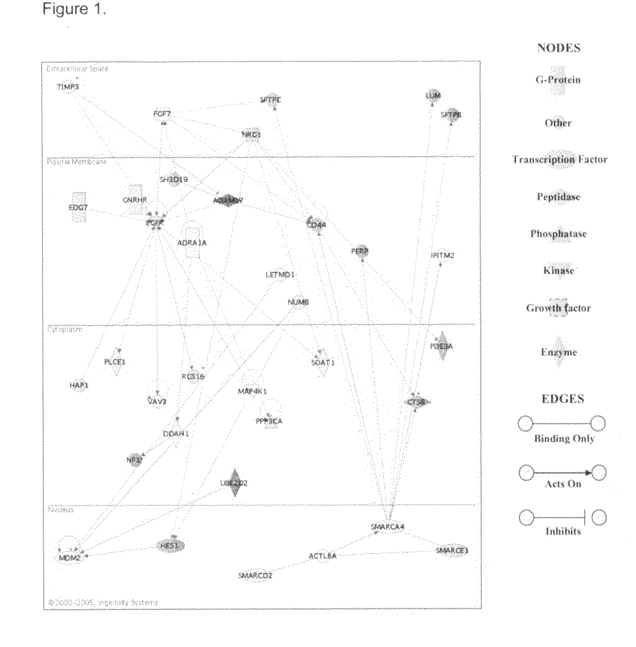

Among these 41 networks, 24 had scores of >8 and the top 2 networks with 35 genes had scores of 49. The top network identified by IPA is associated with nervous system development and function, cellular growth, and proliferation (FIG. 1). Epidermal growth factor receptor (EGFR) is the most outstanding interaction partner found within the network. EGFR interacts with TIMP3, NRG1, ADAM17, EDG7 and FGF7; all are overexpressed, and involved in neural or visual perception development. EGFR signaling is implicated in early events of epidermal, neural and eye development. Loss of EGFR signaling results in reduced brain size and loss of larval eye and optic lobe in drosophila. EGFR expression is required for postnatal forebrain and astrocytes development in mice. Functional pathway analysis conducted on this network using the IPA tool set identified three genes, ADAM17, NUMB and HES1, involved in the Notch signaling pathway which regulates nervous system and eye development. ADAM17 and NUMB were overexpressed while HES1 was repressed in both the groups. This analysis suggests that LCPUFAs influence many processes with influences that converge on EGFR. It further illustrates that DHA and ARA supplementation, according to the method of the present invention, can improve cellular growth and proliferation and nervous system, epidermal, and eye development and function. Thus, a method of the present invention is directed to improving at least one of these areas via a therapeutically effective amount of DHA and ARA supplementation.

LCPUFA are known to directly interact with nutrient sensitive transcription factors such as peroxisome proliferator-activated receptors (PPARs), liver X receptors, hepatic nuclear factor-4α, sterol regulatory binding proteins, retinoid X receptors and NF-KB. Upon ingestion, LCPUFA can elicit a transcriptional response within minutes. Microarray studies on LCPUFA-supplemented animals have identified several tissue-specific pathways regulated by LCPUFA, particularly involving the liver, adipose, and brain tissue transcriptome. Using murine 11K Affymetrix oligoarrays, Berger, et al. showed increased hepatic expression of lipolytic and decreased expression of lipogenic genes. Berger, et al., Unraveling Lipid Metabolism with Microarrays: Effects of Arachidonate and Docosaheaenoate Acid on Murine Hepatic and Hippocampal Gene Expression, Genome Bio. 3(7): preprint00004 (2002); Berger, et al., Dietary Effects of Arachidonate-Rich Fungal Oil and Fish Oil on Murine Hepatic and Hippocampal Gene Expression, Lipids Health Dis. 1(2): 2 (2002).

However, in the hippocampus brain region, increased expression of HTR4 and decreased expression of TTR and SIAT8E, genes involved in the regulation of cognition and learning, as well as POMC, a gene associated with appetite control, was identified. The first paper published on the brain gene transcriptome with respect to LCPUFA supplementation by Kitajka, et al. demonstrated that feeding fish oil (DHA 26.9%) to rats increased expression of genes involved in lipid metabolism (SPTLC2, FPS), energy metabolism (ATP synthase subunit d, ATP synthase H⁺, cytochromes, IDH3G), cytoskeleton (Actin related protein 2, TUBA 1), signal transduction (Calmodulins, SH3P4, RAB6B small GTPase), receptors, ion channels and neurotransmission (Vasopressin V1b receptor, Somatostatin), synaptic plasticity (Synucleins) and regulatory proteins (protein phosphatases). Kitijka, et al., The Role of n-3 Polyunsaturated Fatty Acids in Brain: Modulation of Rat Brain Gene Expression by Dietary n-3 Fatty Acids, Proc. Natl. Acad. Sci. 99(5): 2619-24 (2002).

In the same study, fish oil supplementation also significantly reduced the expression of phospholipase D and Transthyretin. In related work, Kitajka, et al., using rat cDNA microarrays with 3,200 spots, found results similar to those previously reported. Kitajka, et al., Effects of Dietary Omega-3 Polyunsaturated Fatty Acids on Brain Gene Expression, Proc. N. Acad. Sci. 101(30): 10931-10936 (2004). Barcelo-Coblijn, et al. were the first to report moderation of age-induced changes in gene expression in rat brain as a result of diets rich in fish oil (DHA 11.2%). Barcelo-Coblijn, et al., Modification by Docosahexaenoic Acid of Age-Induced Alterations in Gene Expression and Molecular Composition of Rat Brain Phospholipids, Proc. Natl. Acad. Sci. 100(20): 11321-26 (2003). In this study, 2 month old rats showed increased expression of SNCA and TTR, however, 2-year old rats exhibited no significant changes. Id.

In addition, Puskas, et al. demonstrated that administration of omega-3 fatty acids from fish oil (5% EPA and 27% DHA; total fat content: 8%) for 4 weeks in 2-year old rats induced expression of transthyretin and mitochondrial creatine kinase and decreased expression of HSP86, ApoC-l and Makorin RING zinc-finger protein 2, genes in hippocampus brain region. Puskas, et al., Short-Term Administration of Omega 3 Fatty Acids from Fish Oil Results in Increased Transthyretin Transcription in Old Rat Hippocamus, Proc. Natl. Acad. Sci. 100(4): 1580-85 (2003). Finally, Flachs, et al. showed increased expression of genes for mitochondrial proteins in adipose tissue. Flachs, et al., Polyunsaturated Fatty Acids of Marine Origin Upregulate Mitochondrial Biogenesis and Induce Beta-Oxidation in White Fat, Diabetologia 48(11): 2365-2375 (2005).

In comparison with previous brain transcriptome analyses, the present study employing the use of high-density Affymetrix oligoarrays (>54,000 ps) revealed genes differentially regulated by LCPUFA at ranges mimicking breast milk. The present data indicate that LCPUFA supplementation within the ranges of breast milk will induce global changes in gene expression across numerous biological processes.

### CONCLUSIONS

The impact of DHA and ARA on infant baboons was both significant and widespread. Several novel differentially-expressed transcripts were identified in 12-week old baboon cerebral cortexes modulated by dietary LCPUFA. The majority of probe sets showed subtle changes in gene transcription. In the cerebral cortex, increased expression of mitochondrial proton carrier, UCP2 (uncoupling protein 2) was observed in both groups, but more in L3/C. PLA2G6, implicated in childhood neurodegeneration, was differentially expressed. TIA1, a silencer of the COX2 gene translation was upregulated in L3/C. Increased expression was observed for TIMM8A, NRG1, SEMA3D and NUMB, genes involved in neural development. LUM, EML2, TIMP3 and TTC8 genes with roles in visual perception were overexpressed. Hepatic nuclear factor-4α (HNF4A) showed decreased expression with increasing DHA. RARA was repressed in both the groups.

A network involving 35 genes attributed to neural development and function was identified using Ingenuity network analysis, emphasizing EGFR as the most outstanding interaction partner in the network. In this network EGFR interacts with genes involved in neural or visual perception, TIMP3, NRG1, ADAM17, EDG7 and FGF7. Although subtle, the upregulation of NUMB and downregulation of HES1 in the Notch signaling pathway, not previously shown to interact with fatty acids, supports the involvement of LCPUFA, particularly DHA, in neural development. Interestingly, no known desaturases and only one elongase, LCPUFA biosynthetic enzymes, were differentially expressed in cerebral cortex.

In a study of liver gene expression, fatty acid desaturases SCD and FADS1 were significantly downregulated. A multifunctional protein, TOB1, was significantly overexpressed in the liver. TOB1 is a gene that was affected by DHA and ARA supplementation. It is a transducer of ERBB2, 1 and was upregulated in the liver and thymus by 30% in the L group and by 110% in the L3 group, as compared to the control group. TOB1 is a novel multifunctional anti-proliferative protein involved in hippocampus-dependent learning and memory. Jin, et al., The Negative Cell Cycle Regulator, Tob (Transducer of Erb-2), is a Multifunctional Protein Involved in Hippocampus-Dependent Learning and Memory, Neurosc. 131(3):647-59 (2005). The gene has also been linked with the regulation of quiescence in lymphocytes, tumor suppression, and decreased incidences of osteoarthritis. Yusuf and Fruman, Regulation of Quiescence in Lymphocytes, Trends Immunol. 24(7):380-86 (2003); Yoshida, et al., Mice Lacking a Transcriptional Corepressor Tob are Predisposed to Cancer, Genes Dev. 17(10):1201-06 (2003); Gebauer, et al., Repression of Anti-Proliferative Factor Tob1 in Osteoarthritic Cartilige, Arthritis Res. Ther. 7(2):R274-R284 (2005). Thus, because the gene is indicated in connection with learning, memory, tumor suppression, and osteoarthritis, it is believed that upregulation of TOB1 through DHA and ARA supplementation prevents and/or treats each of these functions or disorders.

These data represent the first comprehensive transcriptome analysis in primates and have identified widespread changes in cerebral cortex genes that are modulated by increases in DHA, induced by dietary means. Importantly, the range of DHA used herein is within limits of human and primate breast milks, the natural food for infants, and indicate that CNS gene expression responds to LCPUFA concentrations.

The inventors have determined that increasing levels of DHA and ARA induces the regulation of global changes in gene expression across diverse biological processes. For example, in an embodiment of the present invention, DHA and ARA supplementation is effective in increasing plasma Ceramide and LysoSM levels, tumor suppression, preventing iron related disorders, improving neurological development such as speech, learning and memory, mediating an immune response, increasing lung function and development, and preventing heart, skin, intestinal, and lung abnormalities. The inventors also believe that an embodiment of the present invention is effective in preventing or treating various neurodegenerative disorders, various cancers, such as breast, pancreatic, colorectal, ovarian, endometrial, and prostatic, as well as osteoarthritis, schizophrenia and Alzheimer's disease.

In addition, regulation at the transcription and/or translational levels of genes involved in the lipid machinery, such as absorption, transport, and metabolism, can lead to lower plasma triglyceride levels, lower accumulation of lipids in adipocytes, increased utilization and hydrolysis of triglycerides, and increased fatty acid oxidation in adipocytes and muscles. These actions can orchestrate lowering adiposity, weight gain, and the occurrence of obesity and atherosclerosis in infants and children.

All references cited in this specification, including without limitation, all papers, publications, patents, patent applications, presentations, texts, reports, manuscripts, brochures, books, internet postings, journal articles, periodicals, and the like, are hereby incorporated by reference into this specification in their entireties. The discussion of the references herein is intended merely to summarize the assertions made by their authors and no admission is made that any reference constitutes prior art. Applicants reserve the right to challenge the accuracy and pertinence of the cited references.

Although embodiments of the invention have been described using specific terms, devices, and methods, such description is for illustrative purposes only. The words used are words of description rather than of limitation. It is to be understood that changes and variations may be made by those of ordinary skill in the art without departing from the spirit or the scope of the present invention, which is set forth in the following claims. In addition, it should be understood that aspects of the various embodiments may be interchanged both in whole or in part. For example, while methods for the production of a commercially sterile liquid nutritional supplement made according to those methods have been exemplified, other uses are contemplated. Therefore, the spirit and scope of the appended claims should not be limited to the description of the versions contained therein.

What is claimed is:

1. A method for modulating the expression of one or more genes in a subject, wherein the gene is selected from the group consisting of those genes listed in Tables 4-9 under the “Gene Symbol” column, the method comprising administering to the subject ARA and DHA.

2. The method according to claim 1, wherein the subject is one that is in need of such modulation.

3. The method according to claim 1, wherein the ARA and the DHA are administered to the subject in a ratio of from about 10:1 to about 1:10 by weight.

4. The method according to claim 1, wherein the ARA and the DHA are administered to the subject in a ratio of from about 2:1 to about 1:2 by weight.

5. The method according to claim 1, wherein the ratio of ARA:DHA is about 1:1.5 by weight.

6. The method according to claim 1, wherein the subject is an infant.

7. The method according to claim 6, wherein the amount of DHA administered to the infant is between about 15 mg per kg of body weight per day and 60 mg per kg of body weight per day.

8. The method according to claim 6, wherein the amount of ARA administered to the infant is between about 20 mg per kg of body weight per day and 60 mg per kg of body weight per day.

9. The method according to claim 6, wherein the DHA and ARA are administered to an infant during the time period from birth until the infant is about one year of age.

10. The method according to claim 6, wherein the DHA and ARA are administered to an infant in an infant formula.

11. A method for upregulating the expression of one or more genes in a subject, wherein the gene is selected from the group consisting of those genes listed in Tables 4 and 6 under the “Gene Symbol” column, the method comprising administering to the subject ARA and DHA.

12. The method according to claim 11, wherein the subject is one in need of such upregulation.

13. The method according to claim 11, wherein the subject is a human infant.

14. The method according to claim 11, wherein the ARA and DHA are administered to the subject in a ratio of ARA:DHA of between about 1:2 to about 2:1 by weight.

15. A method for downregulating the expression of one or more genes in a subject, wherein the gene is selected from the group consisting of those genes listed in Tables 5 and 7 under the “Gene Symbol” column, the method comprising administering to the infant ARA and DHA.

16. The method according to claim 15, wherein the subject is one in need of such downregulation.

17. The method according to claim 15, wherein the subject is a human infant.

18. The method according to claim 15, wherein the ARA and DHA are administered to the subject in a ratio of ARA:DHA of between about 1:2 to about 2:1 by weight.

19. A method for upregulating the expression of one or more genes in a subject, wherein the gene is selected from the group consisting of TIMM8A, TIMM23, EGFR, NF1, SFTPB, ACADSB, SOD, PDE3A, NSMAF, OSBP2, FTH1, SPTLC2, FOXP2, LUM, BRCA1, ADAM17, ADAM33, TOB1, XCL1, XCL2, RNASE2, RNASE3, SULT1C1, HSPCA, CD44, CD24, OSBPL9, FCER1G, FXD3, NRF1, STK3, KIR2DS1, and any combination thereof, the method comprising administering to the subject ARA and DHA.

20. The method according to claim 19, wherein the subject is one in need of such upregulation.

21. The method according to claim 19, wherein the subject is a human infant.

22. The method according to claim 19, wherein the ARA and DHA are administered to the subject in a ratio of ARA:DHA of between about 1:2 to about 2:1 by weight.

23. A method for modulating the expression of one or more genes in a subject, wherein the gene is selected from the group consisting of TIMM8A, TIMM23, NF1, LUM, BRCA1, ADAM17, TOB1, RNASE2, RNASE3, NRF1, STK3, FZD3, ADAM8, PERP, COL4A6, PLA2G6, MSRA, CTSD, CTSB, LMX1B, BHMT, TNNC1, PDE3A, PPARD, NPY1R, LEP, and any combination thereof, the method comprising administering to the subject ARA and DHA.

24. A method for treating or preventing tumors in a subject, the method comprising modulating the expression of a gene selected from the group consisting of TOB1, NF1, FZD3, STK3, BRCA1, NRF1, PERP, and COL4A6 in the subject by administering to the subject an effective amount of DHA and ARA.

25. The method according to claim 24, wherein the subject is in need of such modulation.

26. The method according to claim 24, wherein the subject is a human infant.

27. The method according to claim 24, wherein the ARA and DHA are administered to the subject in a ratio of ARA:DHA of between about 1:2 to about 2:1 by weight.

28. A method for treating or preventing neurodegeneration in a subject, the method comprising modulating the expression of a gene selected from the group consisting of PLA2G6, TIMM8A, ADAM17, TIMM23, MSRA, CTSD, CTSB, LMX1B, and BHMT in the subject by administering to the subject an effective amount of DHA and ARA.

29. The method according to claim 28, wherein the neurodegenerative condition treated or prevented is selected from the group consisting of Mohr-Tranebjaerg syndrome, Jensen syndrome, Alzheimer's disease, Parkinson's disease, nail patella syndrome, and congenital ovine neuronal ceroid lipofuscinosis.

30. A method for improving vision in a subject, the method comprising modulating the expression of the LUM gene in the subject by administering to the subject an effective amount of DHA and ARA.

31. A method for treating or preventing macular degeneration in a subject, the method comprising modulating the expression of the LUM gene in the subject by administering to the subject an effective amount of DHA and ARA.

32. The method according to claim 31, wherein the macular degeneration is Sorsby's fundus.

33. A method for stimulating an immune response in a subject, the method comprising modulating the expression of a gene selected from the group consisting of RNASE2, RNASE3, and ADAM8 in the subject by administering to the subject an effective amount of DHA and ARA.

34. A method for improving lung function in a subject, the method comprising modulating the expression of the ADAM33 gene in the subject by administering to the subject an effective amount of DHA and ARA.

35. The method according to claim 34 comprising the treatment or prevention of a disorder selected from the group consisting of asthma, and bronchial hyperresponsiveness.

36. A method for improving cardiac function in a subject, the method comprising modulating the expression of a gene selected from the group consisting of TNNC1 and PDE3A in the subject by administering to the subject an effective amount of DHA and ARA.

37. The method according to claim 36, wherein the idiopathic dilated cardiomyopathy is treated or prevented.

38. A method for treating or preventing obesity in a subject, the method comprising modulating the expression of a gene selected from the group consisting of PPARD, NPY1R, and LEP in the subject by administering to the subject an effective amount of DHA and ARA.

39. The method according to claim 38, wherein the method treats or prevents a disorder selected from the group consisting of hyperglycemia and type II diabetes.

40. A method for modulating the expression of one or more genes in an infant, wherein the gene is selected from the group consisting of those genes listed in Tables 4-9 under the “Gene Symbol” column, the method comprising administering to the infant DHA.

41. The method according to claim 40, wherein the expression is upregulated in a gene selected from the group consisting of those genes listed in Tables 4 and 6 under the “Gene Symbol” column.

42. The method according to claim 40, wherein the expression is downregulated in a gene is selected from the group consisting of those genes listed in Tables 5 and 7 under the “Gene Symbol” column.

43. A method for modulating the expression of one or more genes in an infant, wherein the gene is selected from the group consisting of those genes listed in Tables 4-9 under the “Gene Symbol” column, the method comprising administering to the infant ARA.

44. A method for modulating the expression of one or more genes in a child, wherein the gene is selected from the group consisting of those genes listed in Tables 4-9 under the “Gene Symbol” column, the method comprising administering to the child DHA.

45. The method according to claim 44, wherein the child is between the ages of one and six years of age.

46. The method according to claim 44, wherein the child is between the ages of about seven and twelve years of age.

47. The method according to claim 44 additionally comprising administering ARA to the child.

48. A method for modulating the expression of one or more genes in a child, wherein the gene is selected from the group consisting of TIMM8A, TIMM23, NF1, LUM, BRCA1, ADAM17, TOB1, RNASE2, RNASE3, NRF1, STK3, FZD3, ADAM8, PERP, COL4A6, PLA2G6, MSRA, CTSD, CTSB, LMX1B, BHMT, TNNC1, PDE3A, PPARD, NPY1R, LEP, and any combination thereof, the method comprising administering to the child DHA.

49. The method according to claim 48, wherein the child is between the ages of one and six years of age.

50. The method according to claim 48, wherein the child is between the ages of about seven and twelve years of age.

51. The method according to claim 48 additionally comprising administering ARA to the child.

52. A method for modulating the expression of one or more genes in a child, wherein the gene is selected from the group consisting of those genes listed in Tables 4-9 under the “Gene Symbol” column, the method comprising administering to the child ARA.


SEQUENCE LISTING (25 sequences)

SEQ ID NO: 1 - DNA, 22 bp, Homo sapiens
tgggcaatcatcaccaaactgt

SEQ ID NO: 2 - DNA, 20 bp, Homo sapiens
acatggcacttggtagcttt

SEQ ID NO: 3 - DNA, 17 bp, Homo sapiens
cagggcagttacattct

SEQ ID NO: 4 - DNA, 24 bp, Homo sapiens
tgcaccagatgactgaactttgtt

SEQ ID NO: 5 - DNA, 20 bp, Homo sapiens
agcccgactgtccaactttg

SEQ ID NO: 6 - DNA, 16 bp, Homo sapiens
tccatgcacttctccc

SEQ ID NO: 7 - DNA, 24 bp, Homo sapiens
ccagcacgatgaagatcaagatca

SEQ ID NO: 8 - DNA, 16 bp, Homo sapiens
gccgccgatccacaca

SEQ ID NO: 9 - DNA, 16 bp, Homo sapiens
cctgagcgcaagtact

SEQ ID NO: 10 - DNA, 20 bp, Homo sapiens
accattgcctctgctcttgt

SEQ ID NO: 11 - DNA, 20 bp, Homo sapiens
ttcaaccgcttgcgatgctt

SEQ ID NO: 12 - DNA, 20 bp, Homo sapiens
tgctacttgcaaaggcgtgt

SEQ ID NO: 13 - DNA, 20 bp, Homo sapiens
acccaacgatgttgtctgct

SEQ ID NO: 14 - DNA, 20 bp, Homo sapiens
tgctgcaattgcctgtgtca

SEQ ID NO: 15 - DNA, 20 bp, Homo sapiens
tccacaaccttgcactgctt

SEQ ID NO: 16 - DNA, 20 bp, Homo sapiens
acagcagctttggcaatgga

SEQ ID NO: 17 - DNA, 20 bp, Homo sapiens
aatgtggccgagaggcaaat

SEQ ID NO: 18 - DNA, 20 bp, Homo sapiens
ttgtcaacagggcaaggcaa

SEQ ID NO: 19 - DNA, 20 bp, Homo sapiens
tgggtgcttcaaggccattt

SEQ ID NO: 20 - DNA, 20 bp, Homo sapiens
agcaccgtcaatgcctacaa

SEQ ID NO: 21 - DNA, 20 bp, Homo sapiens
aggcagaagtgaagtggcaa

SEQ ID NO: 22 - DNA, 20 bp, Homo sapiens
tgccaaggcacgagtaacaa

SEQ ID NO: 23 - DNA, 20 bp, Homo sapiens
aggaattcgctccactgtgt

SEQ ID NO: 24 - DNA, 20 bp, Homo sapiens
attgccgacaggatgcagaa

SEQ ID NO: 25 - DNA, 20 bp, Homo sapiens
aagcatttgcggtggacgat